(19) United States
(12) Patent Application Publication
(10) Pub. No.: US 2022/0255013 A1
(43) Pub. Date: Aug. 11, 2022

(54) ORGANIC COMPOUND AND ORGANIC ELECTROLUMINESCENCE DEVICE USING THE SAME

(71) Applicants: FENG-WEN YEN, TAIPEI (TW); TSUN-YUAN HUANG, CHIAYI (TW)

(72) Inventors: FENG-WEN YEN, TAIPEI (TW); TSUN-YUAN HUANG, CHIAYI (TW)

(73) Assignee: LUMINESCENCE TECHNOLOGY CORP., Hsin-Chu (TW)

(21) Appl. No.: 17/248,776

(22) Filed: Feb. 8, 2021

(51) Int. Cl. H01L 51/00; C07D 495/04; C09K 11/06; C07D 519/00; C07D 491/048; C07D 491/14; C07D 491/22

(52) U.S. Cl. CPC H01L 51/0071; C07D 495/04; C09K 11/06; H01L 51/0067; H01L 51/5016; C07D 519/00; C07D 491/048; C07D 491/14; C07D 491/22; H01L 51/0072

(57) ABSTRACT

An organic compound having the following formula (F) is disclosed. An organic electroluminescence device comprises the organic compound as a phosphorescent host, a fluorescent host, a hole blocking layer, or an electron transport layer. The organic compound is for lowering a driving voltage, or increasing a current efficiency or a half-life of an organic electroluminescence device.

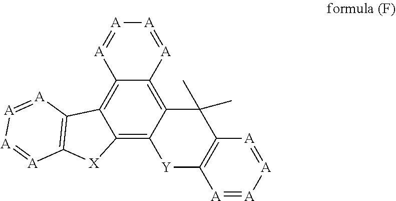

The same definition as described in the present invention.

### FIELD OF INVENTION

The present invention relates to a novel organic compound and, more particularly, to an organic electroluminescence device using the organic compound.

### BACKGROUND OF THE INVENTION

Organic electroluminescence (organic EL) devices, i.e., organic light-emitting diodes (OLEDs) that make use of organic compounds, are becoming increasingly desirable than before. One of the organic compounds has the following formula:

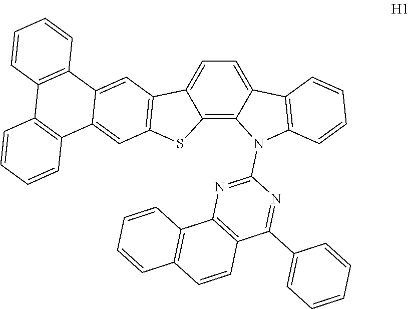 [SMILES: c1ccc(-c2nc(-n3c4ccccc4c4ccc5c6cc7c8ccccc8c8ccccc8c7cc6sc5c43)nc3c2ccc2ccccc23)cc1]

For OLEDs, organic compounds may have performance advantages over conventional materials. For example, the wavelength at which an emissive layer emits light may generally be readily tuned with appropriate dopants. However, there is still a need for improvement of those organic compounds in an organic EL device, for example, in relation to the current efficiency, driving voltage or half-life of the organic EL device.

### SUMMARY OF THE INVENTION

An object of the invention may be to provide an organic compound and an organic EL device using the same.

Another object of the present invention may be to improve an organic compound of an organic EL device, so that the organic EL device may have a higher current efficiency, a lower driving voltage, or a longer half-life.

According to the present invention, an organic compound which can be applied in an organic EL device is disclosed. The organic compound may have the following formula (F):

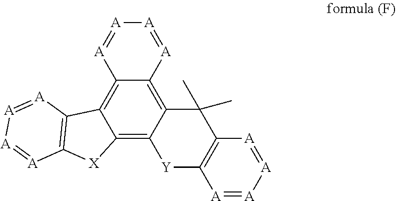

wherein X is O or S if Y is N-L-Z;
wherein Y is O or S if X is N-L-Z;
wherein all A are the same or different at each instance and are independently N or CH;
wherein L represents a single bond or a substituted or unsubstituted divalent arylene group having 6 to 30 ring carbon atoms; and
wherein Z represents a substituted or unsubstituted aryl group having 6 to 60 carbon atoms, or represents a substituted or unsubstituted heteroaryl group having 6 to 60 carbon atoms.

The present invention further discloses an organic EL device. The organic EL device may comprise an anode, a cathode and one or more organic layers formed between the anode and the cathode. At least one of the organic layers comprises the organic compound of formula (F).

### BRIEF DESCRIPTION OF THE DRAWINGS

FIG. 1 is a cross-sectional view of a first organic EL device according to a second embodiment of the present invention.

FIG. 2 is a cross-sectional view of an organic EL device without the host 340C of FIG. 1.

FIG. 3 is a cross-sectional view of a second organic EL device according to a third embodiment of the present invention.

FIG. 4 is a cross-sectional view of a third organic EL device according to a fourth embodiment of the present invention.

### DESCRIPTION OF THE PREFERRED EMBODIMENTS

What probed into the invention is the organic compound and organic EL device using the organic compound. Detailed descriptions of the production, structure and elements will be provided as follows such that the invention can be fully understood. Obviously, the application of the invention is not confined to specific details familiar to those skilled in the art. On the other hand, the common elements and procedures that are known to everyone are not described in details to avoid unnecessary limits of the invention. Some preferred embodiments of the present invention will now be described in greater detail as follows. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

As used herein, the “aza” designation in the fragments described herein, i.e., aza-aromatic hydrocarbons, aza-dibenzofuran, aza-dibenzothiophene, etc. means that one or more of the C—H groups in the respective fragment can be replaced by N (a nitrogen atom). For example, and without any limitation, aza-triphenylene encompasses both dibenzo[fh]quinoxaline and dibenzo[fh]quinoline. One of ordinary skill in the art can readily envision other nitrogen analogs of the aza-derivatives described above, and all such analogs are intended to be encompassed by the terms as set forth herein.

It is to be understood that when a molecular fragment is described as being a substituent or otherwise attached to another moiety, its name may be written as if it were a fragment (e.g., phenyl, phenylene, naphthyl, dibenzofuryl) or as if it were the whole molecule (e.g., benzene, naphthalene, dibenzofuran). As used herein, these different ways of designating a substituent or attached fragment are considered to be equivalent.

As used herein, “combinations thereof” indicates that one or more members of the applicable list are combined to form a known or chemically stable arrangement that one of ordinary skill in the art can envision from the applicable list. For example, benzoquinazoline may be substituted by phenyl or pyridinyl, the substitution is an example of a combination. Moreover, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The term “alkyl” or “alkyl group” refers to and includes both straight and branched chain alkyl radicals. Preferred alkyl groups are those containing from 1 to 20 carbon atoms, more preferably 1 to 15 carbon atoms. Suitable alkyl groups include methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aryl group” refers to and includes both single-ring aromatic hydrocarbonyl groups and polycyclic aromatic ring systems. The polycyclic rings may have two, three, four or more rings in which two carbons are common to two adjoining rings (the rings are “fused”) wherein at least one of the rings is an aromatic hydrocarbonyl group, e.g., the other rings can be cycloalkyls, cycloalkenyls, aryl, heterocycles, and/or heteroaryls. Preferred aryl groups are those containing 6 to 30 carbon atoms, preferably 6 to 20 carbon atoms, more preferably 6 to 12 carbon atoms. Especially preferred is an aryl group having 6 carbons, 10 carbons or 12 carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, and naphthalene. Additionally, the aryl group is optionally substituted.

The terms “aralkyl”, “aralkyl group” or “arylalkyl” are used interchangeably and refer to an alkyl group that is substituted with an aryl group. Preferred aralkyl groups are those containing 6 to 30 carbon atoms. Additionally, the aralkyl group is optionally substituted.

The term “heteroaryl” or “heteroaryl group” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing 3 to 30 carbon atoms, preferably 3 to 20 carbon atoms, more preferably 3 to 12 carbon atoms. Suitable heteroaryl groups include pyrimidine, triazine, quinazoline, benzoquinazoline, phenylquinazoline, dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrazine, oxazine, oxathiazine, oxadiazine, indole, benzimidazole, indazole, indoxazine, benzoxazole, benzisoxazole, benzothiazole, quinoline, isoquinoline, cinnoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

In a first embodiment of the present invention, an organic compound, also a heteroacene, may have the following formula (F):

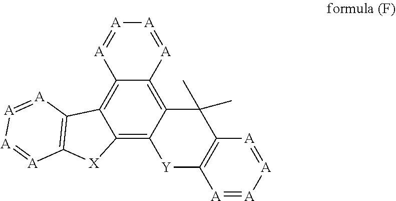

wherein X is O or S if Y is N-L-Z;
wherein Y is O or S if X is N-L-Z;
wherein all A are the same or different at each instance and are independently N or CH;
wherein L represents a single bond or a substituted or unsubstituted divalent arylene group having 6 to 30 ring carbon atoms; and
wherein Z represents a substituted or unsubstituted aryl group having 6 to 60 carbon atoms, or represents a substituted or unsubstituted heteroaryl group having 6 to 60 carbon atoms.

In an organic EL device, the organic compound of formula (F) may be a phosphorescent host or a fluorescent host of an emissive layer. The organic compound of formula (F) may also be an electron transport material (ETM) to form an electron transport layer (ETL), or a hole blocking material (HBM) to form a hole blocking layer (HBL) in an organic EL device.

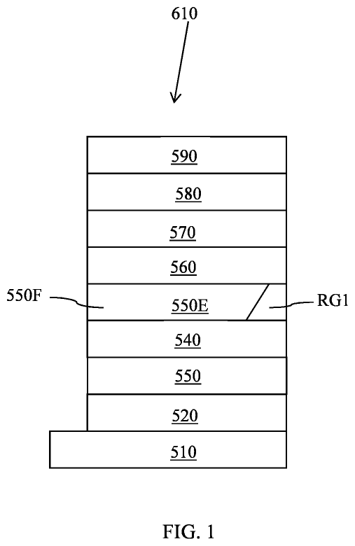

In a second embodiment of the present invention, a first organic EL device using the organic compound of formula (F) is disclosed. FIG. 1 is a cross-sectional view of the first organic EL device. Referring to FIG. 1, the first organic EL device 610 may comprise the organic compound of formula (F) as a host 550F of an emissive layer 550E.

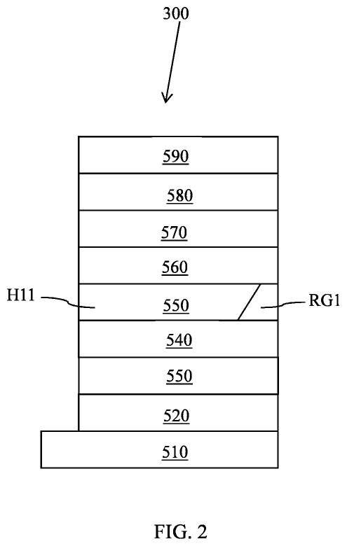

FIG. 2 is a cross-sectional view of an organic EL device without the organic compound of formula (F) (without 550F of FIG. 1). Referring to FIG. 2, the organic EL device 300 may have a driving voltage of about 4.5 V, a current efficiency of about 17.3 cd/A, or a half-life of about 820 hours.

Referring to FIG. 1, by comprising the organic compound of formula (F) as the host 550C, the first organic EL device 610 may have a driving voltage lower than that of the organic EL device 300 (FIG. 2). Moreover, by comprising the organic compound of formula (F) as the host 550C, the first organic EL device 610 of FIG. 1 may have a current efficiency higher than that of the organic EL device 300 (FIG. 2). Furthermore, by comprising the organic compound of formula (F) as the host 550C, the first organic EL device 610 of FIG. 1 may have a half-life longer than that of the organic EL device 300 (FIG. 2).

Still referring to FIG. 1, as the host 550C of the first organic EL device 610 of FIG. 1, the organic compound of formula (F) may lower the driving voltage to be about 3.3 V to about 4.3 V. Moreover, the organic compound of formula (F) may increase the current efficiency to be 21.4 cd/A to about 27.1 cd/A. Furthermore, the organic compound of formula (F) may increase the half-life to be about 910 hours to about 1580 hours.

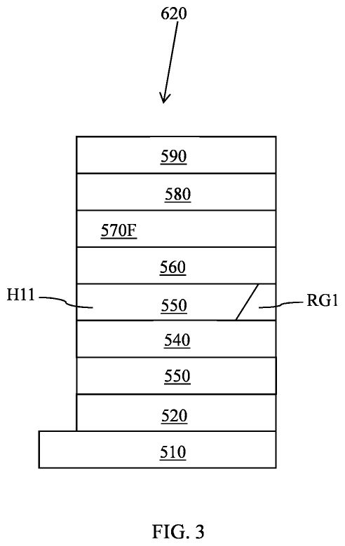

FIG. 3 is a cross-sectional view of the second organic EL device in a third embodiment of the present invention. Referring to FIG. 3, a second organic EL device 620 using the organic compound of formula (F) is disclosed. The second organic EL device 620 may comprise the organic compound of formula (F) as an electron transport layer 570F.

FIG. 2 is a cross-sectional view of an organic EL device without the organic compound of formula (F) (without 570F of FIG. 3). Referring to FIG. 2, the organic EL device 300 may have a driving voltage of about 4.5 V, a current efficiency of about 17.3 cd/A, or a half-life of about 820 hours.

Referring to FIG. 3, by comprising the organic compound of formula (F) as the electron transport layer 570F, the second organic EL device 620 may have a driving voltage lower than that of the organic EL device 300 (FIG. 2). Moreover, by comprising the organic compound of formula (F) as electron transport layer 570F, the second organic EL device 620 of FIG. 3 may have a current efficiency higher than that of the organic EL device 300 (FIG. 2). Furthermore, by comprising the organic compound of formula (F) as the electron transport layer 570F, the second organic EL device 520 of FIG. 3 may have a half-life longer than that of the organic EL device 300 (FIG. 2).

Referring to FIG. 3, as the electron transport layer 570F of the second organic EL device 620, the organic compound of formula (F) may lower the driving voltage to be about 3.8 V to about 4.5 V. Moreover, the organic compound of formula (F) may increase the current efficiency to be about 17.5 cd/A to about 19.1 cd/A. Furthermore, the organic compound of formula (F) may increase the half-life to be about 840 hours to about 980 hours.

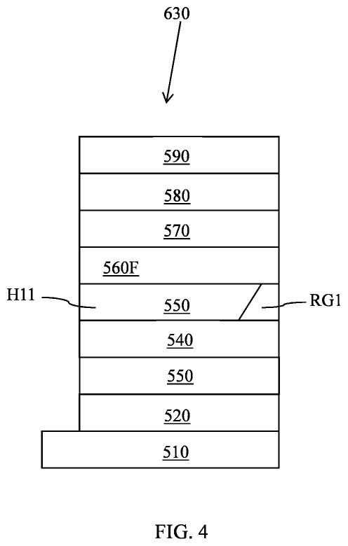

FIG. 2 is a cross-sectional view of an organic EL device without the organic compound of formula (F) (without 560F of FIG. 4). Referring to FIG. 2, the organic EL device 300 may have a driving voltage of about 4.5 V, a current efficiency of about 17.3 cd/A, or a half-life of about 820 hours.

Referring to FIG. 4, by comprising the organic compound of formula (F) as the hole blocking layer 560F, the third organic EL device 630 may have a driving voltage lower than that of the organic EL device 300 (FIG. 2). Moreover, by comprising the organic compound of formula (F) as the hole blocking layer 560F, the second organic EL device 620 of FIG. 3 may have a current efficiency higher than that of the organic EL device 300 (FIG. 2). Furthermore, by comprising the organic compound of formula (F) as the e hole blocking layer 560F, the second organic EL device 520 of FIG. 3 may have a half-life longer than that of the organic EL device 300 (FIG. 2).

Referring to FIG. 3, as the hole blocking layer 560F of the third organic EL device 630, the organic compound of formula (F) may lower the driving voltage to be bout 4.0 V to about 4.4 V. Moreover, the organic compound of formula (F) may increase the current efficiency to be about 17.6 cd/A to about 18.7 cd/A. Furthermore, the organic compound of formula (F) may increase the half-life to be about 830 hours to about 930 hours.

In formula (F), Z is selected from the group consisting of the following:

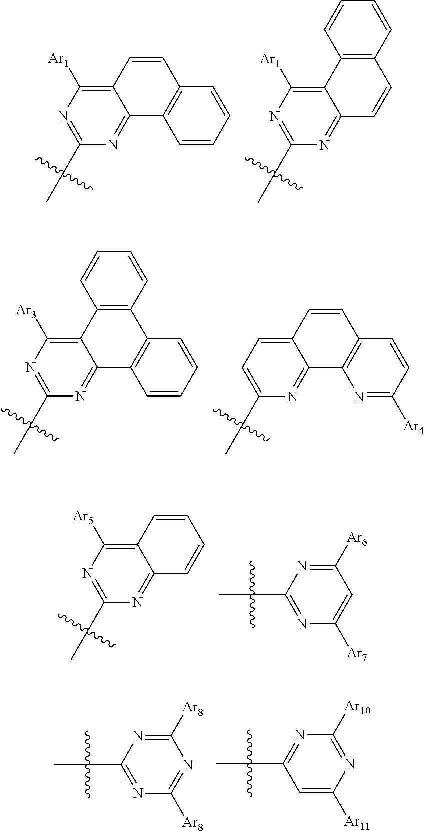

. Ar₁ to Ar₁₁ may independently represent a hydrogen atom, a substituted or unsubstituted aryl group having 6 to 30 carbon atoms, or a substituted or unsubstituted heteroaryl group having 6 to 30 carbon atoms.

In formula (F), Z may be selected from the group consisting of the following:

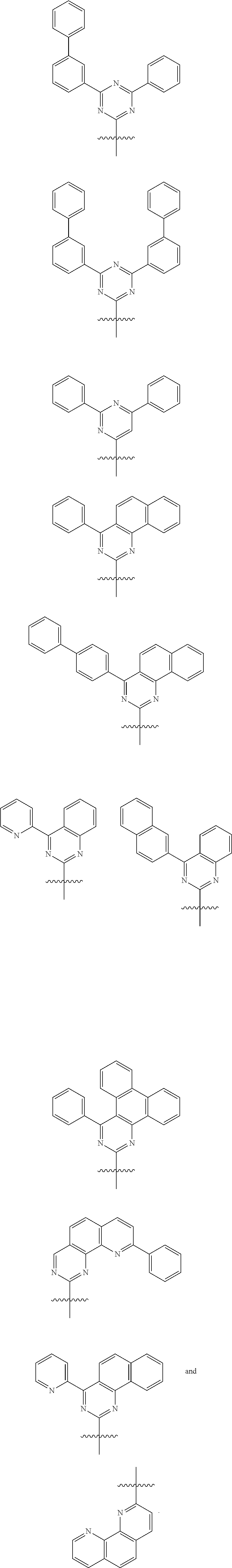

Alternatively, the organic compound may have the following formula (I):

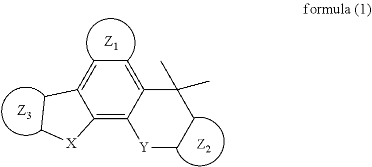

wherein X is O or S if Y is N-L-Z₄; Y is O or S if X is N-L-Z₄;
wherein L represents a single bond or arylene;
wherein Z₁ to Z₃ independently represent aromatic hydrocarbons, or aza-aromatic hydrocarbons; and
wherein Z₄ is substituted or unsubstituted heteroaryl having at least two heteroatoms of N.

The heteroaryl of Z₄ may be selected from the group consisting of benzoquinazolinyl, triazinyl, diazinyl, dibenzoquinazolinyl, pyridinyl, phenylpyridinyl, quinazolinyl, benzodiazinyl, pyridinoquinolinyl, phenyl, biphenyl, and combinations thereof. Preferably, The heteroaryl may be substituted by phenyl, biphenyl, dibenzofuranyl, dibenzothiophenyl, 9,9-dimethyl-9H-fluorenyl, naphthalenyl, or combinations thereof.

Z₄ may be polycyclic heteroaryl or monocyclic heteroaryl, wherein the polycyclic heteroaryl has a plurality of aromatic rings. Z₄ may be heteroaryl having only two heteroatoms of N. Both of the only two heteroatoms of N may preferably in one of the aromatic rings. The polycyclic heteroaryl may be tricyclic heteroaryl or tetracyclic heteroaryl. Each of Z₁ to Z₃ may preferably represent benzene. If one of Z₁ to Z₃ is aza-aromatic hydrocarbons, the aza-aromatic hydrocarbons is preferably heteroarene having only one heteroatom of N.

The organic compound of the present invention may be selected from the group consisting of the following:

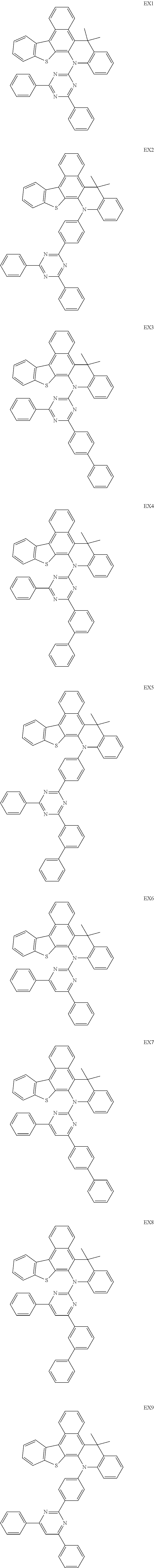

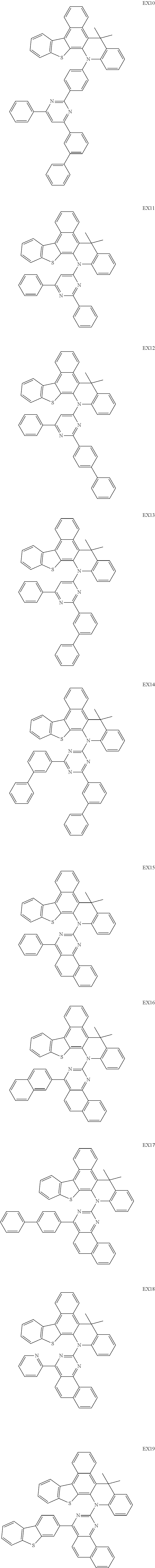

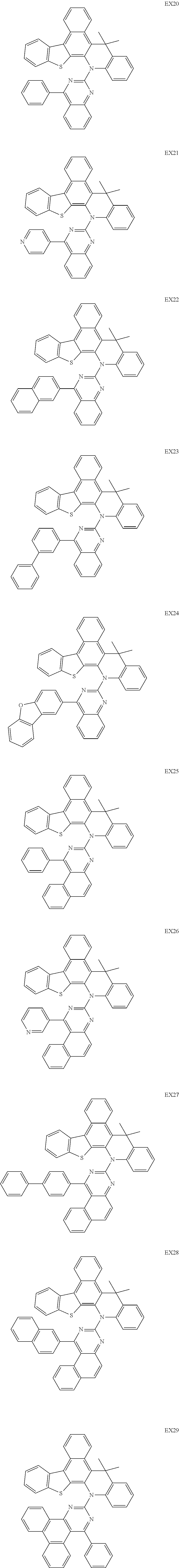

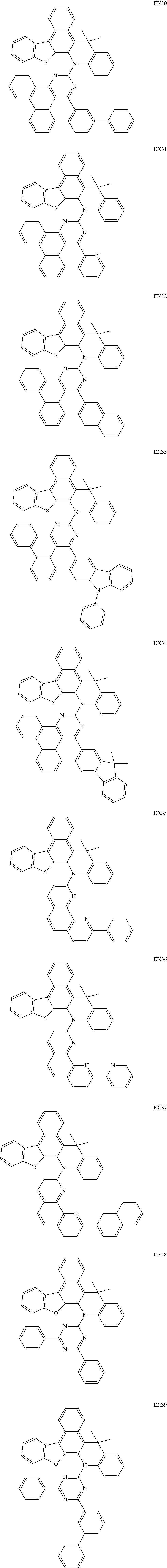

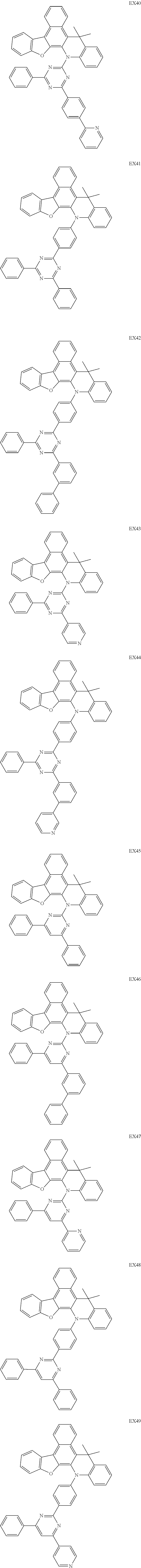

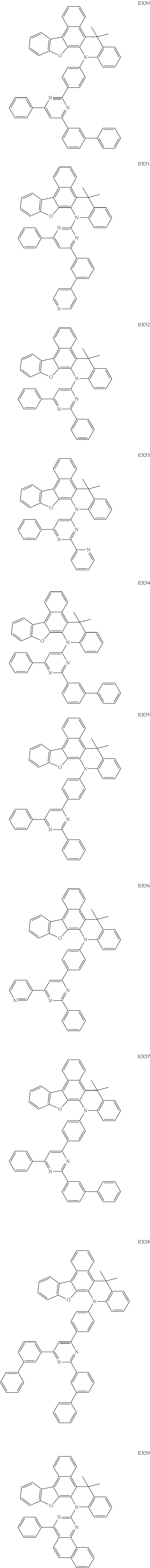

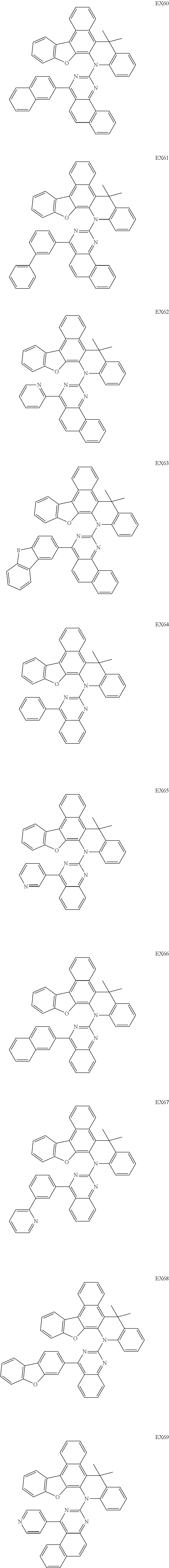

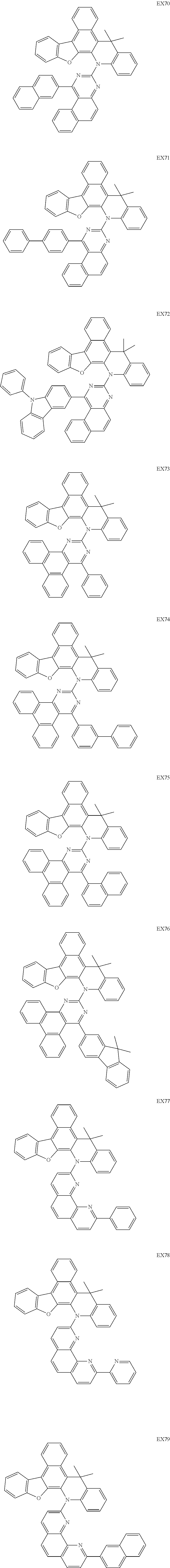

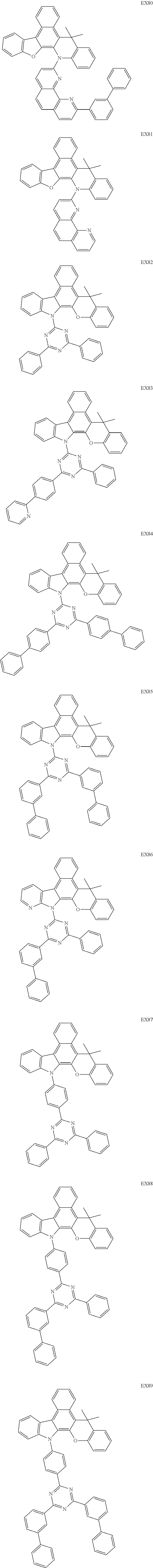

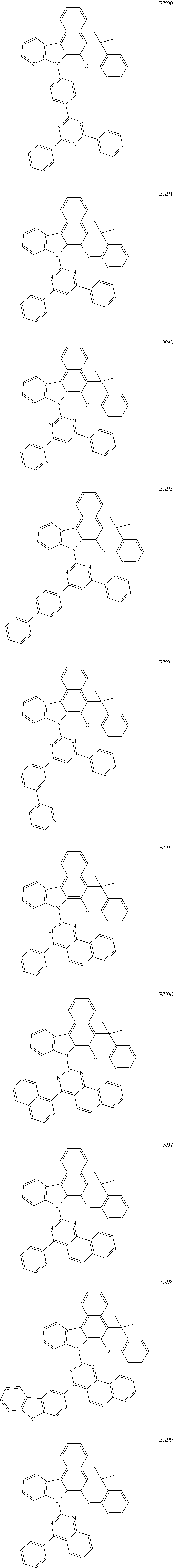

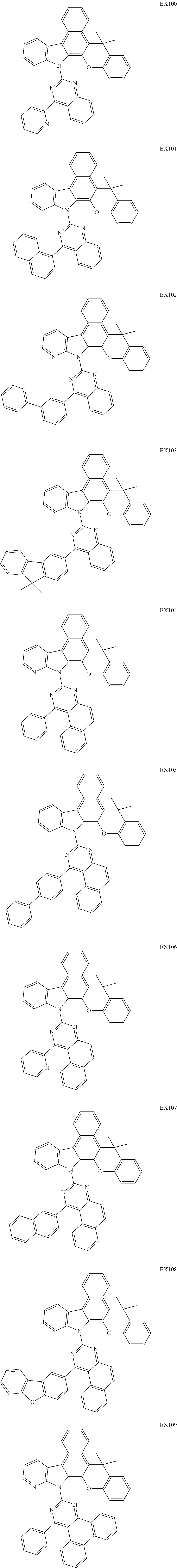

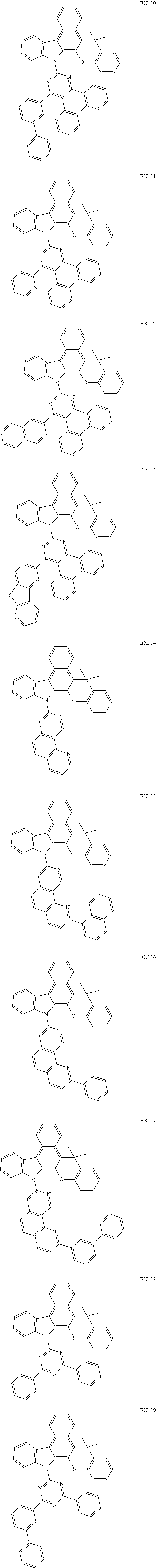

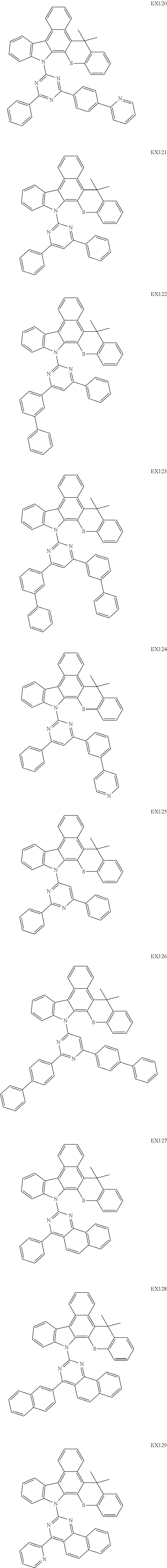

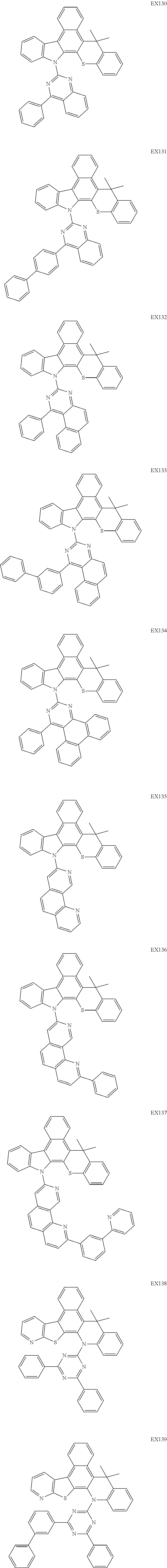

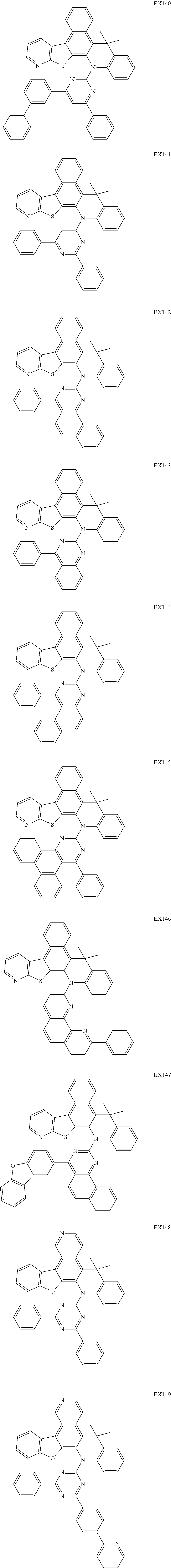

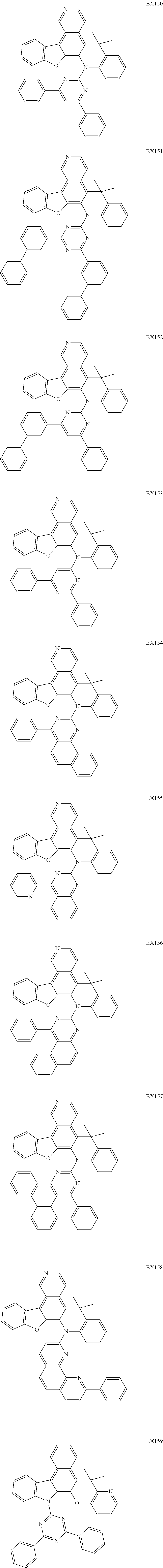

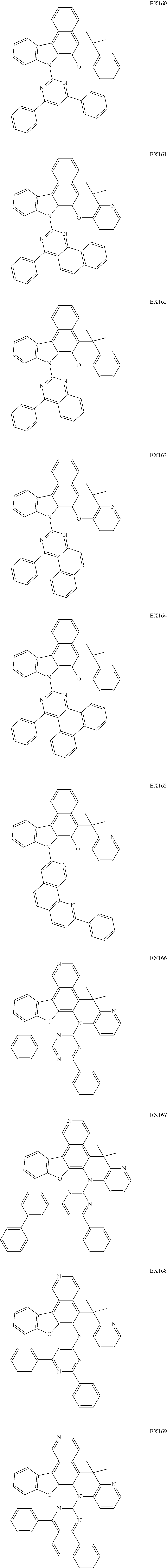

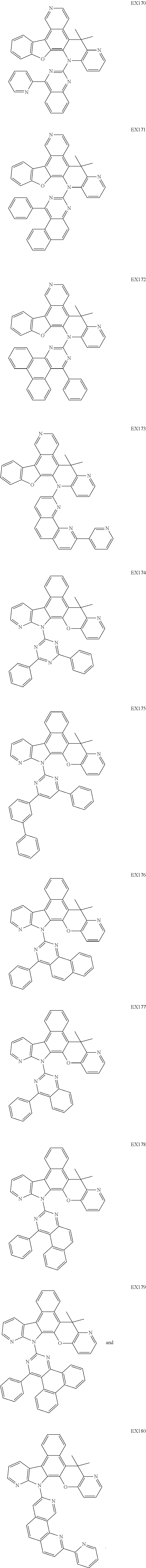

Referring to FIG. 1, the first organic EL device 610 may comprise an anode 510, a cathode 590 and one or more organic layers 520, 530, 540, 550E, 560, 570, 580 formed between the anode 510 and the cathode 589. From the bottom to the top, the one or more organic layers may comprise a hole injection layer 520, a hole transport layer 530, an electron blocking layer 540, an emissive layer 550E, a hole blocking layer 560, an electron transport layer 570 and an electron injection layer 580.

The emissive layer 550E may comprise a 15% dopant RG1 and the organic compound of formula (F) 550F doped with the dopant RG1. The dopant RG1 may be a red guest material for tuning the wavelength at which the emissive layer 550E emits light, so that the color of emitted light may be red. The organic compound of formula (F) may be a host 550F of the emissive layer 550E.

FIG. 2 is a cross-sectional view of an organic EL device without the organic compound of formula (F). Referring to FIG. 2, the organic EL device 300 may comprise an anode 510, a cathode 590 and one or more organic layers 520, 530, 540, 550, 560, 570, 580 formed between the anode 510 and the cathode 590. From the bottom to the top, the one or more organic layers may comprise a hole injection layer 320, a hole transport layer 330, an electron blocking layer 540, an emissive layer 550, a hole blocking layer 560, an electron transport layer 570 and an electron injection layer 580. The emissive layer 550 may comprise a 15% dopant RG1 and an organic compound H11 doped with the dopant RG1. The dopant RG1 may be a red guest material. The organic compound (H11 in paragraph [0002]) may be a host H11 of the emissive layer 550.

To those organic EL devices of FIG. 1 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 1 and FIG. 2 may be summarized in Table 1 below. The half-life is defined as the time that the initial luminance of 1000 cd/m² has dropped to half.

TABLE 1

| | | Driving | Current | | |
|---|---|---|---|---|---|
| Host | | Voltage | Efficiency | Device | Half-life |
| (H11 or 550F) | Dopant | (V) | (cd/A) | Color | (hours) |
| H11 | RG1 | 4.5 | 17.3 | red | 820 |
| EX4 | RG1 | 3.6 | 25.1 | red | 1360 |
| EX13 | RG1 | 3.5 | 25.8 | red | 1430 |
| EX15 | RG1 | 3.3 | 27.1 | red | 1580 |
| EX25 | RG1 | 3.3 | 26.9 | red | 1550 |
| EX29 | RG1 | 3.7 | 24.1 | red | 1230 |
| EX45 | RG1 | 3.6 | 24.9 | red | 1340 |
| EX59 | RG1 | 3.4 | 26.2 | red | 1490 |
| EX73 | RG1 | 3.7 | 24.0 | red | 1210 |
| EX82 | RG1 | 3.6 | 24.6 | red | 1310 |
| EX86 | RG1 | 3.7 | 24.3 | red | 1280 |
| EX95 | RG1 | 3.5 | 26.0 | red | 1470 |
| EX104 | RG1 | 3.9 | 23.0 | red | 1060 |
| EX118 | RG1 | 3.5 | 25.3 | red | 1370 |
| EX127 | RG1 | 3.4 | 25.5 | red | 1390 |
| EX140 | RG1 | 3.8 | 23.8 | red | 1190 |
| EX144 | RG1 | 4.3 | 21.4 | red | 910 |
| EX148 | RG1 | 4.0 | 23.1 | red | 1080 |
| EX154 | RG1 | 3.9 | 23.6 | red | 1130 |
| EX161 | RG1 | 3.9 | 23.5 | red | 1110 |
| EX167 | RG1 | 4.1 | 22.7 | red | 1020 |
| EX171 | RG1 | 4.1 | 22.5 | red | 990 |
| EX176 | RG1 | 4.2 | 22.1 | red | 980 |

According to Table 1, in the first organic EL device 610, the organic compound of formula (F) comprised as a host 550F of FIG. 1 exhibits performance better than a prior art organic EL material (H11).

A method of producing the first organic EL device 610 of FIG. 1 and the organic EL device 300 of FIG. 2 is described. ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water).

Before vapor deposition of the organic layers, cleaned ITO substrates may be further treated by UV and ozone. All pre-treatment processes for ITO substrate are under clean room (class 100), so that an anode 510 may be formed.

One or more organic layers 320, 330, 340 (FIG. 1), 340E (FIG. 1), 350, 360, 370 are applied onto the anode 310 in order by vapor deposition in a high-vacuum unit (10⁻⁷ Torr), such as resistively heated quartz boats. The thickness of the respective layer and the vapor deposition rate (0.1-0.3 nm/sec) are precisely monitored or set with the aid of a quartz-crystal monitor. It is also possible, as described above, each of the organic layers may comprise more than one organic compound. For example, an emissive layer 550E or 550 may be formed of a dopant and a host doped with the dopant. An emissive layer 550E or 550 may also be formed of a co-host and a host co-deposited with the co-host. This may be successfully achieved by co-vaporization from two or more sources. Accordingly, the compounds for the organic layers of the present invention are thermally stable.

Referring to FIG. 1 and FIG. 2, onto the anode 510, Dipyrazino [2,3-f:2,3-] quinoxaline-2,3,6 ,7,10,11-hexacarbonitrile (HAT-CN) may be applied to form a hole injection layer 520 having a thickness of about 20 nm in the organic EL device 510 or 400. N4,N4,N4′,N4′-tetra([1,1′-biphenyl]-4-yl)-[1,1′-biphenyl]-4,4′-diamine (HT1) may be applied to form a hole transport layer 530 having a thickness of about 170 nm.
N-(9,9′-spirobi[fluoren]-4-yl)-N-([1,1′-biphenyl]-2-yl) -14,14-dimethyl-14H-indeno[1,2-b]triphenylen-12-amine(EB3) may be applied to form an electron blocking layer 540.

Referring to FIG. 1 and FIG. 2, in the organic EL device 610 (FIG. 1) or 300 (FIG. 2), an emissive layer 550E or 550 may be formed to have a thickness of about 30 nm.

Referring to FIG. 2, in the organic EL device 300, a compound H11 of paragraph [0002] may be applied to form a host H11 of an emissive layer 550 of FIG. 2. The emissive layer 550 may further comprise a dopant RG1 as a red guest of the emissive layer 550. On the emissive layer 550, a compound HB3 may be used as a hole blocking material (HBM) to form a hole blocking layer 560.
2-ethyl-1-(4-(10-(naphthalen-2-yl)anthracen-9-yl)phenyl)-1H-benzo[d]imidazol-e (ET2) may be applied as an electron transport material to co-deposit with 8-hydroxyquinolato-lithium (LiQ) at a ratio of about, for example, 1:1, thereby forming an electron transport layer (ETL) 570 of the organic EL device 610 or 300.

Referring to FIG. 1 and FIG. 2, the organic EL device 610 or 300 may further comprise a low work function metal, such as Al, Mg, Ca, Li or K, as a cathode 590 by thermal evaporation. A low work function metal may help electrons injecting the electron transport layer 570 from cathode 590. Between the cathode 590 and the electron transport layer 570, a thin electron injecting layer 580 of LiQ may be introduced, to reduce the electron injection barrier and to improve the performance of the organic EL device 610 or 300. The material of the electron injecting layer 580 may alternatively be metal halide or metal oxide with low work function, such as LiF, MgO, or Li₂O.

The organic compounds ET2, LiQ, RG1, HB3, EB3, H11, HAT-CN and HT1 for producing the organic EL device 300 or 610 in this invention may receptively have the following formulas:

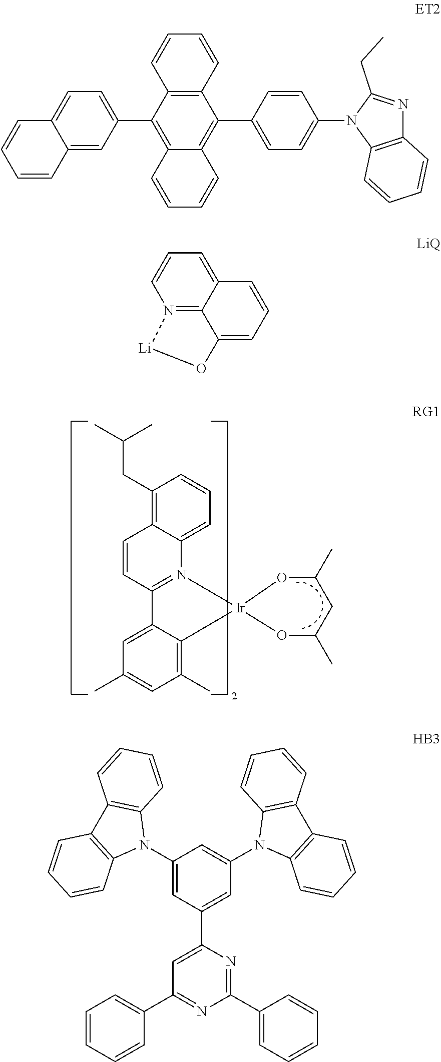

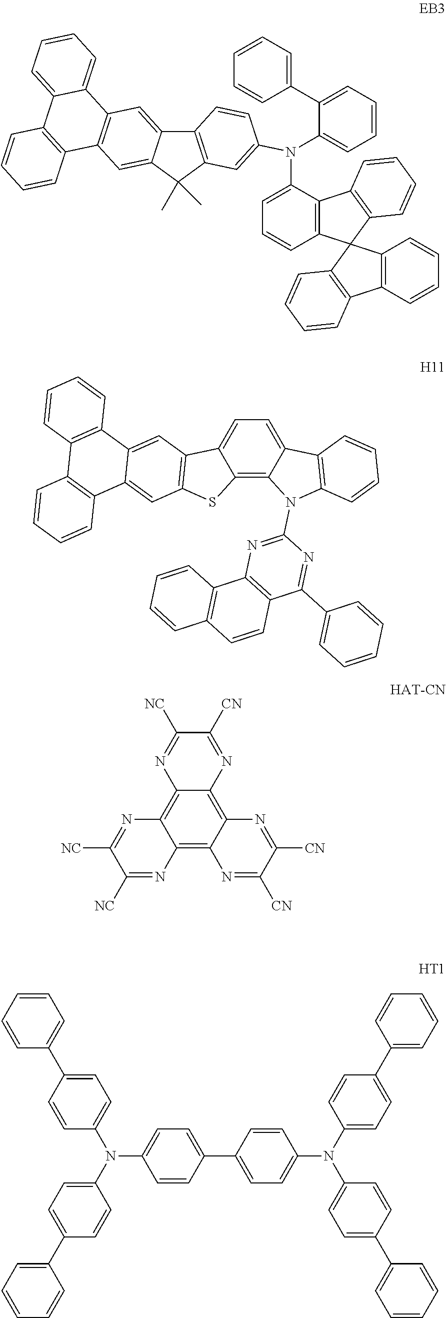 [SMILES: CC1(C)c2cc(N(c3ccccc3-c3ccccc3)c3cccc4c3-c3ccccc3C43c4ccccc4-c4ccccc43)ccc2-c2cc3c4ccccc4c4ccccc4c3cc21 ; c1ccc(-c2nc(-n3c4ccccc4c4ccc5c6cc7c8ccccc8c8ccccc8c7cc6sc5c43)nc3c2ccc2ccccc23)cc1 ; N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

FIG. 3 is a cross-sectional view of the second organic EL device in a third embodiment of the present invention. Referring to FIG. 3, a second organic EL device 620 using the organic compound of formula (F) is disclosed. The method of producing the second organic EL device 620 of FIG. 3 is substantially the same as the method of producing the organic EL device 300 of FIG. 2. The difference is that the electron transport layer 570F of FIG. 3 is made by using the organic compound of formula (F) as an electron transport material (ETM), rather than by using ET2 as an ETM.

To those organic EL devices of FIG. 3 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 3 and FIG. 2 may be summarized in Table 2 below. The half-life of the red-emitting organic EL device 620 or 300 is defined as the time that the initial luminance of 1000 cd/m² has dropped to half.

TABLE 2

| Material for | Driving | Current | Device | Half-life |
|---|---|---|---|---|
| ETL 570 or 570F | Voltage (V) | Efficiency (cd/A) | Color | (hours) |
| ET2 | 4.5 | 17.3 | red | 820 |
| EX35 | 4.0 | 18.5 | red | 910 |
| EX77 | 3.8 | 19.1 | red | 980 |
| EX114 | 3.9 | 18.7 | red | 950 |
| EX146 | 4.3 | 17.6 | red | 850 |
| EX165 | 4.2 | 17.9 | red | 870 |
| EX180 | 4.5 | 17.5 | red | 840 |

According to Table 2, in the second organic EL device 620, the organic compound of formula (F) comprised as an electron transport layer 570F of FIG. 3 exhibits performance better than a prior art electron transport material (ET2 as an ETL 570 of FIG. 2).

FIG. 4 is a cross-sectional view of the third organic EL device in a fourth embodiment of the present invention. Referring to FIG. 4, a third organic EL device 630 using the organic compound of formula (F) is disclosed. The method of producing the second organic EL device 630 of FIG. 4 is substantially the same as the method of producing the organic EL device 300 of FIG. 2. The difference is that the hole blocking layer 560F of FIG. 3 is made by using the organic compound of formula (F) as a hole blocking material (HBM), rather than by using HB3 as a HBM.

To those organic EL devices of FIG. 4 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 4 and FIG. 2 may be summarized in Table 3 below. The half-life of the red-emitting organic EL device 630 or 300 is defined as the time that the initial luminance of 1000 cd/m² has dropped to half.

TABLE 3

| Material for | Driving | Current | Device | Half-life |
|---|---|---|---|---|
| HBL 560 or 560F | Voltage (V) | Efficiency (cd/A) | Color | (hours) |
| HB3 | 4.5 | 17.3 | red | 820 |
| EX13 | 4.1 | 18.4 | red | 910 |
| EX45 | 4.0 | 18.7 | red | 930 |
| EX77 | 4.3 | 17.7 | red | 850 |
| EX82 | 4.1 | 18.1 | red | 880 |
| EX114 | 4.4 | 17.6 | red | 830 |

According to Table 3, in the third organic EL device 630, the organic compound of formula (F) comprised as a hole blocking layer 560F of FIG. 4 exhibits performance better than a prior art hole blocking material (HB3 as an HBL 560 of FIG. 2).

Referring to FIGS. 1, 3 and 4, the organic EL device 610, 620 or 630 of the present invention may alternatively be a lighting panel or a backlight panel.

Detailed preparation of the organic compounds of the present invention will be clarified by exemplary embodiments below, but the present invention is not limited thereto. EXAMPLES 1 to 22 show the preparation of the organic compounds of the present invention.

### EXAMPLE 1

### Synthesis of EX4

### Synthesis of Intermediate 1a

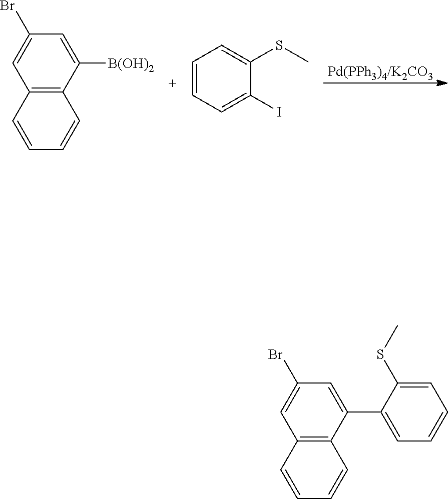

A mixture of (40.0 g, 159.4 mmole) of 3-Bromonaphthalene-1-boronic acid, (36.2 g, 144.9 mmole) of 2-Iodothioanisole, (5.0 g, 4.3 mmole) of Pd(PPh₃)₄, (1.8 g, 4.3 mmole) of 2-Dicyclophosphine-2′,6′-dimethoxybiphenyl, (30.0 g, 217.3 mmole) of K₂CO₃, 720 ml of Toluene and 240 ml of Ethanol, and 110 ml of H₂O was placed under nitrogen, and then heated at 80° C. while stirring for 16 h. After the reaction finished, the mixture was allowed to cool to room temperature. The solution was extracted with 200 ml of ethyl acetate (3 times) and then 300 ml of water. The organic layer was dried with anhydrous magnesium sulfate, and then the solvent was evaporated under reduced pressure. The residue was purified by column chromatography on silica to give product (36.7 g, 77%) as a white solid.

### Synthesis of Intermediate 1b to 1d

Synthesis of Intermediate 1b to 1d was prepared according to the synthesis method of Intermediate 1a.

| Reactant structure | | Product structure | Weight Yield |
|---|---|---|---|
| 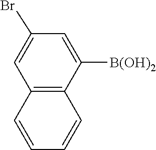 [SMILES: OB(O)c1cc(Br)cc2ccccc12] (20.0 g, 79.7 mmol) | 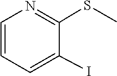 (18.2 g, 72.4 mmol) | 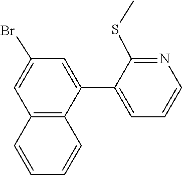 [SMILES: CSc1ncccc1-c1cc(Br)cc2ccccc12] 1b | 23.9g 75% |
| 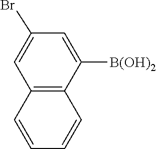 [SMILES: OB(O)c1cc(Br)cc2ccccc12] | 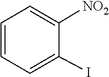 (18.0 g, 72.4 mmol) | 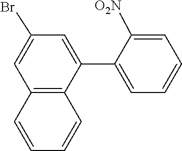 [SMILES: O=[N+]([O-])c1ccccc1-c1cc(Br)cc2ccccc12] | 18.8 g 79% |
| (20.0 g, 79.7 mmol) | | 1c | |
| 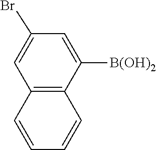 [SMILES: OB(O)c1cc(Br)cc2ccccc12] | 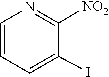 (18.1 g, 72.4 mmol) | 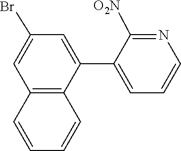 [SMILES: O=[N+]([O-])c1ncccc1-c1cc(Br)cc2ccccc12] | 16.4 g 69% |
| (20.0 g, 79.7 mmol) | | 1d | |

### Synthesis of Intermediate 2a

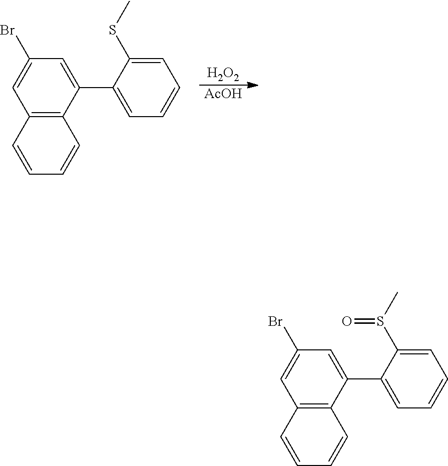

A mixture of Intermediate 1a (36.7 g, 115.6 mmol), 50 ml of DCM and 360 ml of Glacial acetic. To the mixture, 10 ml of 35% H₂O₂ solution was added at 0° C. and the mixture was stirred for 18 h. The solution was extracted with Na₂SO₃ solution. The organic layer was dried with anhydrous magnesium sulfate and then the solvent was evaporated under reduced pressure, yielding Intermediate 2a (39.5 g, 99%) as a off-white solid.

### Synthesis of Intermediate 2b

Synthesis of Intermediate 2b were according to the synthesis method of Intermediate 2a.

| | | Weight |
|---|---|---|
| Reactant structure | Product structure | Yield |
| 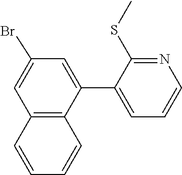 [SMILES: CSc1ncccc1-c1cc(Br)cc2ccccc12] | 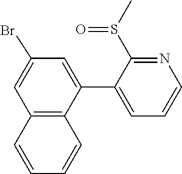 [SMILES: CS(=O)c1ncccc1-c1cc(Br)cc2ccccc12] | 24.5g 98% |
| 1b (23.9 g, 72.4 mmol) | 2b | |

### Synthesis of Intermediate 3a

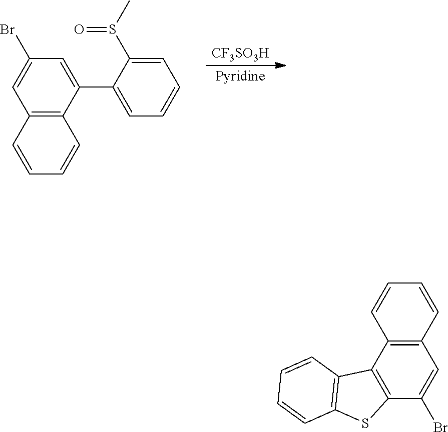 [SMILES: CS(=O)c1ccccc1-c1cc(Br)cc2ccccc12 ; "CF3SO3H" ; "Pyridine" ; Brc1cc2ccccc2c2c1sc1ccccc12]

A mixture of (39.5 g, 114.4 mmol) of Intermediate 2a, (515.1 g, 3432 mol) of Trifluoromethanesulphonic acid was degassed and placed under nitrogen, and then cooled at 5° C. for 48 h. After the reaction finished, 800 ml of water/pyridine 5:1 was added and then heated under reflux for 20 min. After the reaction finished, the mixture was allowed to cool to room temperature. The solution was extracted with dichloromethane and water. The organic layer was dried with anhydrous magnesium sulfate and then the solvent was evaporated under reduced pressure. The pure product is obtained by recrystallization from chlorobenzene to afford Intermediate 3a (28.3 g, 79%) as a light yellow solid.

### Synthesis of Intermediate 3b

Synthesis of Intermediate 3b were according to the synthesis method of Intermediate 3a.

| | | Weight |
|---|---|---|
| Reactant structure | Product structure | Yield |
| 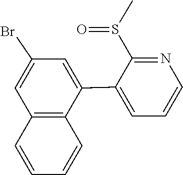 [SMILES: CS(=O)c1ncccc1-c1cc(Br)cc2ccccc12] 2b (24.5 g, 70.8 mmol) | 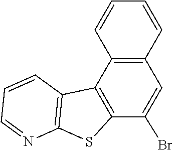 [SMILES: Brc1cc2ccccc2c2c1sc1ncccc12] 3b | 16.5 g 74% |

Synthesis of Intermediate 4a

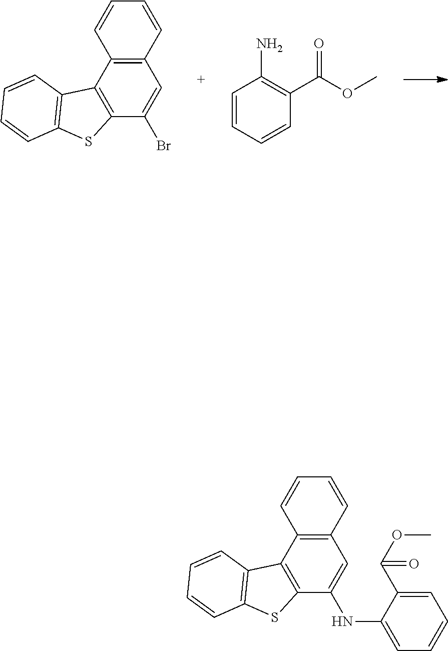 [SMILES: Brc1cc2ccccc2c2c1sc1ccccc12 ; COC(=O)c1ccccc1N ; COC(=O)c1ccccc1Nc1cc2ccccc2c2c1sc1ccccc12]

A mixture of (28.3 g, 90.3 mmole) of Intermediate 3a, (15.0 g, 99.4 mmol) of Methyl 2-aminobenzoate, (0.6 g, 2.7 mmol) of Pd(OAc)₂, (13.0 g, 135.5 mmol) of Sodium tert-butoxide, (1.5 g, 7.2 mmol) of Tri-t-butylphosphine, and 210 ml of Toluene was degassed and placed under nitrogen, and then heated at 110° C. for 16 h. After the reaction finished, the mixture was allowed to cool to room temperature. The solution was extracted with dichloromethane and water. The organic layer was dried with anhydrous magnesium sulfate and then the solvent was evaporated under reduced pressure. The residue was purified by column chromatography on silica to give product (23.5 g, 68%) as a light yellow solid.

### Synthesis of Intermediate 4b

Synthesis of Intermediate 4b were according to the synthesis method of Intermediate 4a.

| | | Weight |
|---|---|---|
| Reactant structure | Product structure | Yield |
| 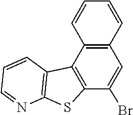 [SMILES: Brc1cc2ccccc2c2c1sc1ncccc12] 3b (16.5 g, 52.5 mmol) | 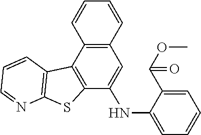 [SMILES: COC(=O)c1ccccc1Nc1cc2ccccc2c2c1sc1ncccc12] 4b | 12.1 g 60% |

### Synthesis of Intermediate 5a

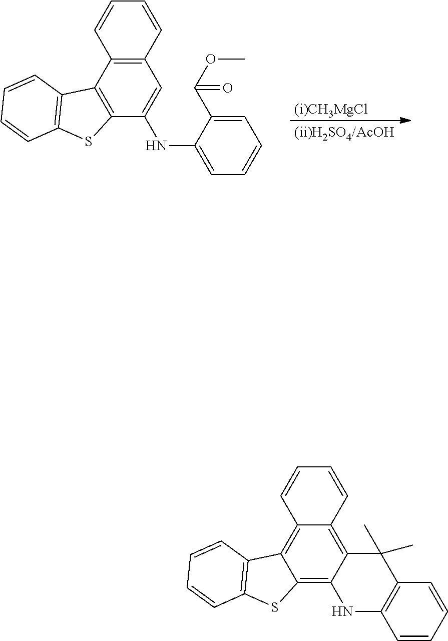

The compound Intermediate 4a (23.5 g, 61.3 mmole) was mixed with 350 ml of dry THF. To the mixture, (23 ml, 183.9 mmole) of 3M CH₃MgBr solution was added at 0° C. and the mixture was stirred for 16 h. The solution was extracted with ethyl acetate and water. The organic layer was dried with anhydrous magnesium sulfate and then the solvent was evaporated under reduced pressure. The residue was mixed with 100 ml of AcOH and 5 ml H₂SO₄ was placed under nitrogen, and then heated at 110° C. while stirring for 12 h. After the reaction finished, the mixture was allowed to cool to room temperature. The solution was extracted with 200 ml of ethyl acetate and then 300 ml of water. The organic layer was dried with anhydrous magnesium sulfate, and then the solvent was evaporated under reduced pressure. The residue was purified by column chromatography on silica to give product (11.8 g, 53%) as a light yellow solid.

### Synthesis of Intermediate 5b

Synthesis of Intermediate 5b were according to the synthesis method of Intermediate 5a.

| | | Weight |
|---|---|---|
| Reactant structure | Product structure | Yield |
| 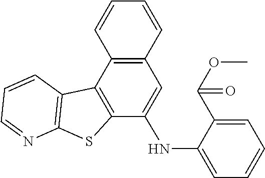 [SMILES: COC(=O)c1ccccc1Nc1cc2ccccc2c2c1sc1ncccc12] | 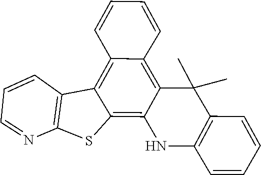 [SMILES: CC1(C)c2ccccc2Nc2c1c1ccccc1c1c2sc2ncccc21] | 5.8 g 50% |
| 4b (12.1 g, 31.5 mmol) | 5b | |

### Synthesis of EX4

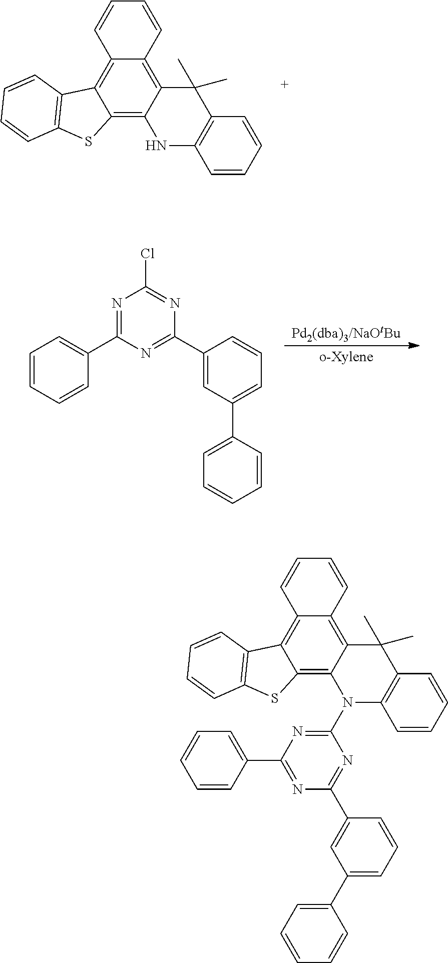 [SMILES: CC1(C)c2ccccc2Nc2c1c1ccccc1c1c2sc2ccccc21 ; Clc1nc(-c2ccccc2)nc(-c2cccc(-c3ccccc3)c2)n1 ; "Pd2(dba)3/NaOtBu" ; "o-Xylene" ; CC1(C)c2ccccc2N(c2nc(-c3ccccc3)nc(-c3cccc(-c4ccccc4)c3)n2)c2c1c1ccccc1c1c2sc2ccccc21]

A mixture of (3.0 g, 8.2 mmole) of Intermediate 5a, (3.4 g, 9.8 mmole) of 2-([1,1′-Biphenyl]-3-yl)-4-chloro-6-phenyl-1,3,5-triazine, (0.37 g, 0.4 mmole) of Pd₂(dba)₃, (1.6 g, 16.4 mmole) of Sodium tert-butoxide, and 30 ml of o-Xylene was degassed and placed under nitrogen, and then heated at 150° C. for 16 h. After the reaction finished, the mixture was allowed to cool to room temperature. Then 300 ml of MeOH was added while stirring and the precipitated product was filtered off with suction to give (3.2 g , 58%) of yellow product, which was recrystallized from EtOH. MS(m/z , EI⁺):671.2

### EXAMPLE 2 to 7

Synthesis of Compound EX13, EX15, EX29, EX140, EX144 and EX146.

Synthesis of Compound EX13, EX15, EX29, EX140, EX144 and EX146 were prepared according to the synthesis method of Compound EX1.

| | | | Weight |
|---|---|---|---|
| Reactant structure | | Product structure | Yield |
| 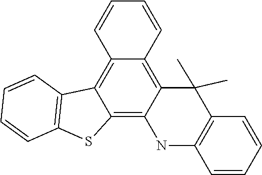 5a (3.0 g, 8.2 mmol) | 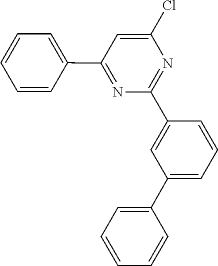 [SMILES: Clc1cc(-c2ccccc2)nc(-c2cccc(-c3ccccc3)c2)n1] (3.4 g, 9.8 mmol) | 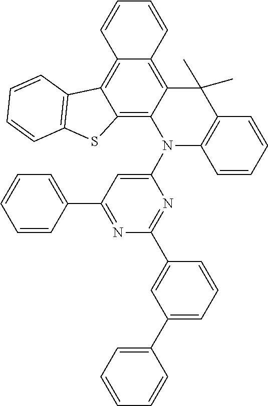 [SMILES: CC1(C)c2ccccc2N(c2cc(-c3ccccc3)nc(-c3cccc(-c4ccccc4)c3)n2)c2c1c1ccccc1c1c2sc2ccccc21] | 2.7 g 49% |
| | | EX13 | |
| | | MS (m/z, EI⁺): 670.7 | |
| 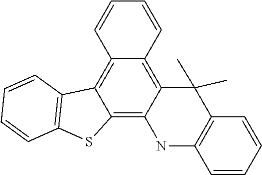 5a (3.0 g, 8.2 mmol) | 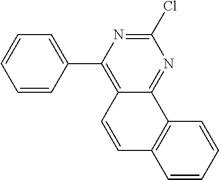 [SMILES: Clc1nc(-c2ccccc2)c2ccc3ccccc3c2n1] (2.9 g, 9.8 mmol) | 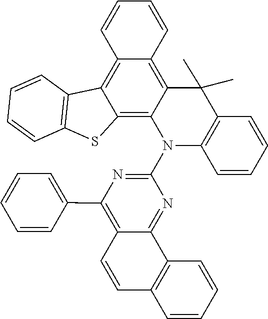 [SMILES: CC1(C)c2ccccc2N(c2nc(-c3ccccc3)c3ccc4ccccc4c3n2)c2c1c1ccccc1c1c2sc2ccccc21] | 2.8 g 56% |
| | | EX15 | |
| | | MS (m/z, EI⁺): 618.8 | |
| 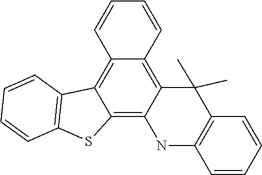 5a (3.0 g, 8.2 mmol) | 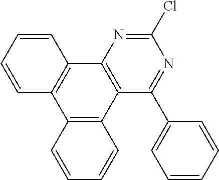 [SMILES: Clc1nc(-c2ccccc2)c2c3ccccc3c3ccccc3c2n1] (3.3 g, 9.8 mmol) | 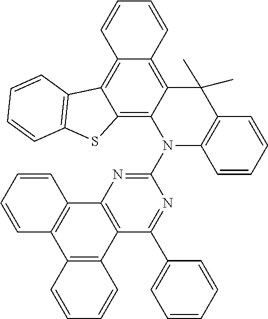 [SMILES: CC1(C)c2ccccc2N(c2nc(-c3ccccc3)c3c4ccccc4c4ccccc4c3n2)c2c1c1ccccc1c1c2sc2ccccc21] | 3.1 g 55% |
| | | EX29 | |
| | | MS (m/z, EI⁺): 698.9 | |
| 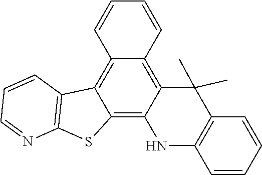 [SMILES: CC1(C)c2ccccc2Nc2c1c1ccccc1c1c2sc2ncccc21] 5b (3.0 g, 8.2 mmol) | 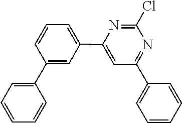 [SMILES: Clc1nc(-c2ccccc2)cc(-c2cccc(-c3ccccc3)c2)n1] (3.4 g, 9.8 mmol) | 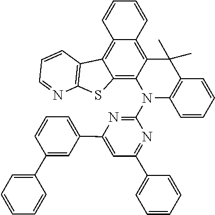 [SMILES: CC1(C)c2ccccc2N(c2nc(-c3ccccc3)cc(-c3cccc(-c4ccccc4)c3)n2)c2c1c1ccccc1c1c2sc2ncccc21] | 3.2 59% |
| | | EX140 | |
| | | MS (m/z, EI⁺): 671.9 | |
| 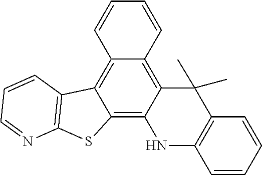 [SMILES: CC1(C)c2ccccc2Nc2c1c1ccccc1c1c2sc2ncccc21] 5b (3.0 g, 8.2 mmol) | 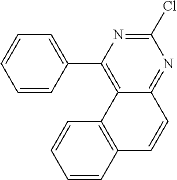 [SMILES: Clc1nc(-c2ccccc2)c2c(ccc3ccccc32)n1] (2.8 g, 9.8 mmol) | 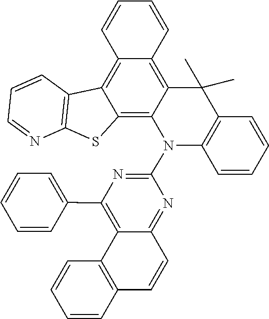 [SMILES: CC1(C)c2ccccc2N(c2nc(-c3ccccc3)c3c(ccc4ccccc43)n2)c2c1c1ccccc1c1c2sc2ncccc21] | 2.6 g 51% |
| | | EX144 | |
| | | MS (m/z, EI⁺): 619.8 | |
| 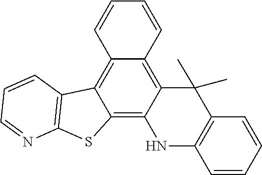 [SMILES: CC1(C)c2ccccc2Nc2c1c1ccccc1c1c2sc2ncccc21] 5b (3.0 g, 8.2 mmol) | 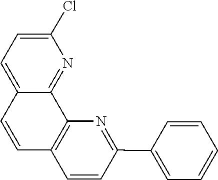 [SMILES: Clc1ccc2ccc3ccc(-c4ccccc4)nc3c2n1] (2.8 g, 9.8 mmol) | 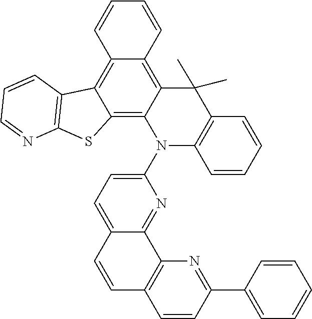 [SMILES: CC1(C)c2ccccc2N(c2ccc3ccc4ccc(-c5ccccc5)nc4c3n2)c2c1c1ccccc1c1c2sc2ncccc21] | 2.4 g 47% |
| | | EX146 | |
| | | MS (m/z, EI⁺): 619.7 | |

### EXAMPLE 8

### Synthesis of EX45

### Synthesis of Intermediate 1e

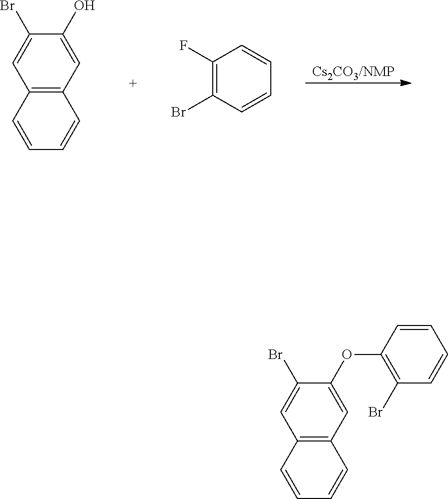 [SMILES: Oc1cc2ccccc2cc1Br ; Fc1ccccc1Br ; "Cs2CO3/NMP" ; Brc1ccccc1Oc1cc2ccccc2cc1Br]

A mixture of (30.0 g, 134.5 mmole) of 3-Bromo-2-naphthol, (25.9 g, 148.0 mmole) of 1-Bromo-2-fluorobenzene, (57.0 g, 174.8 mmole) of Cesium carbonate, 90 ml of N-methyl-2-pyrrolidone was degassed and placed under nitrogen, and then heated at 120° C. overnight. After the reaction finished, the mixture was allowed to cool to room temperature. Then 400 ml of H₂O was added while stirring and the precipitated product was filtered off with suction to give (45.7 g, 90%) of yellow product, which was recrystallized from Toluene.

### Synthesis of Intermediate 1f

Synthesis of Intermediate 1f were according to the synthesis method of Intermediate 1e.

| | | | Weight |
|---|---|---|---|
| Reactant structure | | Product structure | Yield |
| 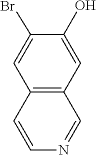 [SMILES: Oc1cc2cnccc2cc1Br] | 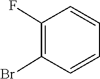 (17.1 g, 98.1 mmol)) | 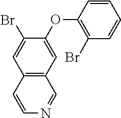 [SMILES: Brc1ccccc1Oc1cc2cnccc2cc1Br] 1f | 29.7 g 88% |
| (20.0 g, 89.2 mmol) | | | |

### Synthesis of Intermediate 3e

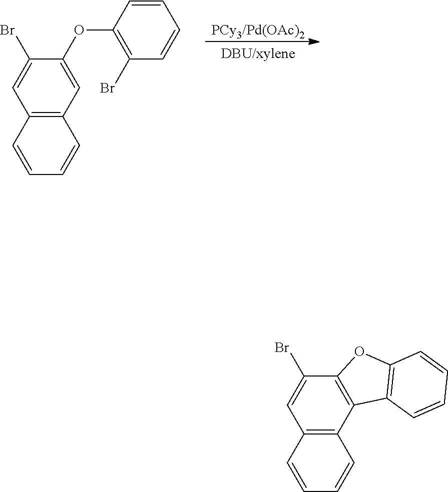 [SMILES: Brc1ccccc1Oc1cc2ccccc2cc1Br ; "DBU/xylene" ; "PCy3/Pd(OAc)2" ; Brc1cc2ccccc2c2c1oc1ccccc12]

A mixture of (45.7 g, 120.9 mmole) of Intermediate 1e, (22.0 g, 145.0 mmole) of 1,8-diazabicyclo(5.4.0)undec-7-ene, (1.4 g, 4.8 mmole) of Tricyclohexylphosphine, (0.5 g, 2.4 mmole) of Pd(OAc)₂, 220 ml of O-xylene was degassed and placed under nitrogen, and then heated at 135° C. for 72 hr. After the reaction finished, the mixture was allowed to cool to room temperature. The solution was extracted with 400 ml of ethyl acetate and then 300 ml of water. The organic layer was dried with anhydrous magnesium sulfate, and then the solvent was evaporated under reduced pressure. The residue was purified by column chromatography on silica to give product (26.2 g, 73%) as a yellow solid.

### Synthesis of Intermediate 3f

Synthesis of Intermediate 3f were according to the synthesis method of Intermediate 3e.

| | | Weight |
|---|---|---|
| Reactant structure | Product structure | Yield |
| 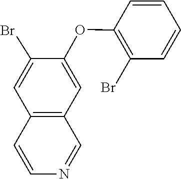 [SMILES: Brc1ccccc1Oc1cc2cnccc2cc1Br] 1f ( 29.7g, 78.3 mmol) | 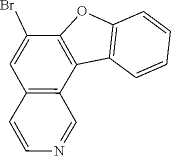 [SMILES: Brc1cc2ccncc2c2c1oc1ccccc12] 3f | 17.5g 75% |

### Synthesis of Intermediate 4e to 4g

Synthesis of Intermediate 4e to 4g were according to the synthesis method of Intermediate 4a.

| | | | Weight |
|---|---|---|---|
| Reactant structure | | Product structure | Yield |
| 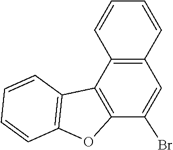 [SMILES: Brc1cc2ccccc2c2c1oc1ccccc12] (26.2 g, 88.2 mmol) | 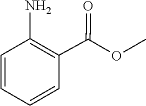 (14.7 g, 97.0 mmol) | 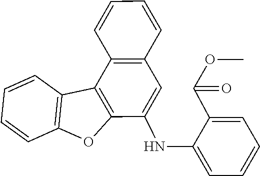 [SMILES: COC(=O)c1ccccc1Nc1cc2ccccc2c2c1oc1ccccc12] 4e | 18.5 g 57% |
| 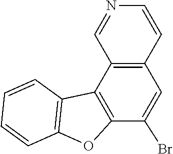 [SMILES: Brc1cc2ccncc2c2c1oc1ccccc12] (17.5 g, 58.7 mmol) | 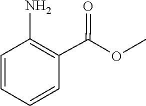 (9.8 g, 64.6 mmol) | 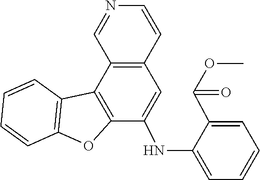 [SMILES: COC(=O)c1ccccc1Nc1cc2ccncc2c2c1oc1ccccc12] 4f | 13.6 g 63% |
| 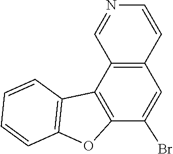 [SMILES: Brc1cc2ccncc2c2c1oc1ccccc12] (20.0 g, 67.1 mmol) | 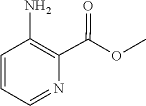 (11.2 g, 73.8 mmol) | 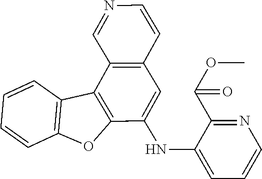 [SMILES: COC(=O)c1ncccc1Nc1cc2ccncc2c2c1oc1ccccc12] 4g | 15.1 g 61 % |

### Synthesis of Intermediate 5e to 5g

Synthesis of Intermediate 5e to 5g were according to the synthesis method of Intermediate 5a.

| | | Weight |
|---|---|---|
| Reactant structure | Product structure | Yield |
| 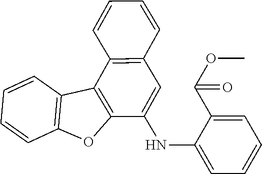 [SMILES: COC(=O)c1ccccc1Nc1cc2ccccc2c2c1oc1ccccc12] | 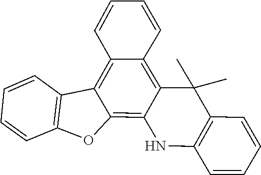 [SMILES: CC1(C)c2ccccc2Nc2c1c1ccccc1c1c2oc2ccccc21] | 10.7 g 61% |
| 4e (18.5 g, 50.3 mmol) | 5e | |
| 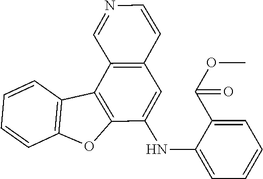 [SMILES: COC(=O)c1ccccc1Nc1cc2ccncc2c2c1oc1ccccc12] | 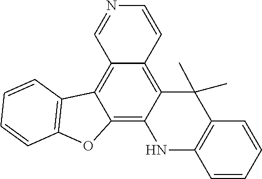 [SMILES: CC1(C)c2ccccc2Nc2c1c1ccncc1c1c2oc2ccccc21] | 7.4 g 57% |
| 4f (13.6 g, 37.0 mmol) | 5e | |
| 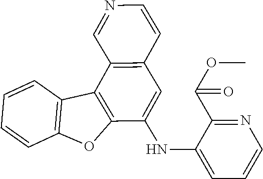 [SMILES: COC(=O)c1ncccc1Nc1cc2ccncc2c2c1oc1ccccc12] | 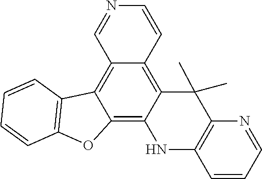 [SMILES: CC1(C)c2ncccc2Nc2c1c1ccncc1c1c2oc2ccccc21] | 7.0 g 49% |
| 4g (15.1 g, 40.9 mmol) | 5g | |

### EXAMPLE 8 to 15

Synthesis of Compound EX45, EX59, EX73, EX77, EX148, EX154, EX167 and EX171.

Synthesis of Compound EX45, EX59, EX73, EX77, EX148, EX154, EX167 and EX171 were prepared according to the synthesis method of Compound EX1.

| | | | Weight |
|---|---|---|---|
| Reactant structure | | Product structure | Yield |
| 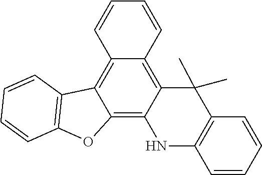 [SMILES: CC1(C)c2ccccc2Nc2c1c1ccccc1c1c2oc2ccccc21] 5e(3.0 g, 8.6 mmol) | 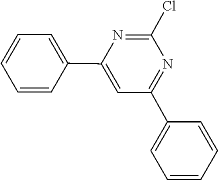 [SMILES: Clc1nc(-c2ccccc2)cc(-c2ccccc2)n1] (2.7 g, 10.3 mmol) | 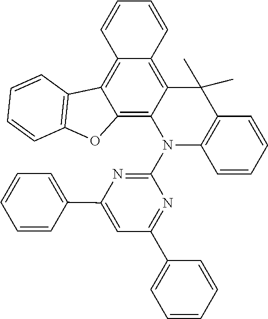 [SMILES: CC1(C)c2ccccc2N(c2nc(-c3ccccc3)cc(-c3ccccc3)n2)c2c1c1ccccc1c1c2oc2ccccc21] | 3.1 g 63% |
| | | EX45 | |
| | | MS (m/z, EI⁺): 578.7 | |
| 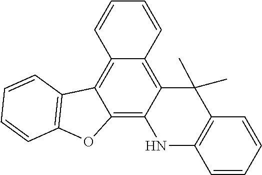 [SMILES: CC1(C)c2ccccc2Nc2c1c1ccccc1c1c2oc2ccccc21] 5e(3.0 g, 8.6 mmol) | 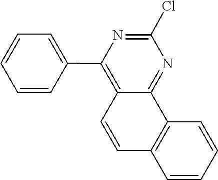 [SMILES: Clc1nc(-c2ccccc2)c2ccc3ccccc3c2n1] (3.0 g, 10.3 mmol) | 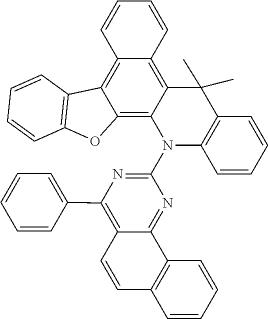 [SMILES: CC1(C)c2ccccc2N(c2nc(-c3ccccc3)c3ccc4ccccc4c3n2)c2c1c1ccccc1c1c2oc2ccccc21] | 3.1 g 61% |
| | | EX59 | |
| | | MS (m/z, EI⁺) : 602.8 | |
| 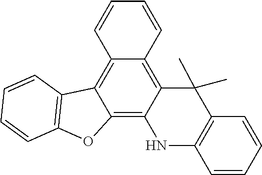 [SMILES: CC1(C)c2ccccc2Nc2c1c1ccccc1c1c2oc2ccccc21] 5e(3.0 g, 8.6 mmol) | 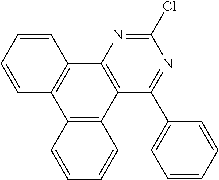 [SMILES: Clc1nc(-c2ccccc2)c2c3ccccc3c3ccccc3c2n1] (3.5 g, 10.3 mmol) | 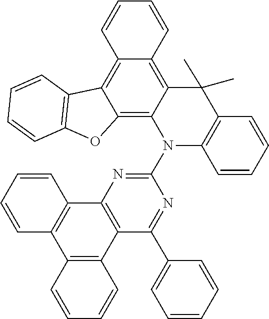 [SMILES: CC1(C)c2ccccc2N(c2nc(-c3ccccc3)c3c4ccccc4c4ccccc4c3n2)c2c1c1ccccc1c1c2oc2ccccc21] | 3.3 g 59% |
| | | EX73 | |
| | | MS (m/z, EI⁺): 652.8 | |
| 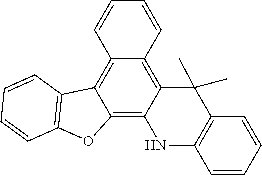 [SMILES: CC1(C)c2ccccc2Nc2c1c1ccccc1c1c2oc2ccccc21] 5e(3.0 g, 8.6 mmol) | 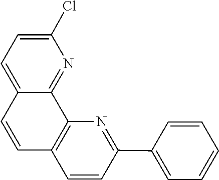 [SMILES: Clc1ccc2ccc3ccc(-c4ccccc4)nc3c2n1] (3.0 g, 10.3 mmol) | 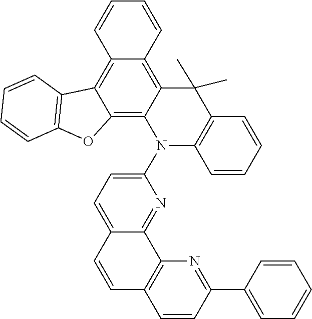 [SMILES: CC1(C)c2ccccc2N(c2ccc3ccc4ccc(-c5ccccc5)nc4c3n2)c2c1c1ccccc1c1c2oc2ccccc21] | 3.1 g 60% |
| | | EX77 | |
| | | MS (m/z, EI⁺): 602.7 | |
| 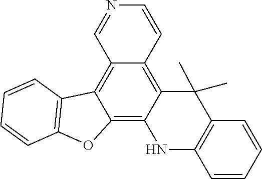 [SMILES: CC1(C)c2ccccc2Nc2c1c1ccncc1c1c2oc2ccccc21] 5f (3.0 g, 8.5 mmol) | 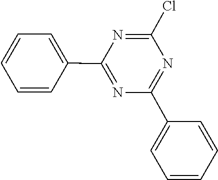 [SMILES: Clc1nc(-c2ccccc2)nc(-c2ccccc2)n1] (2.7 g, 10.2 mmol) | 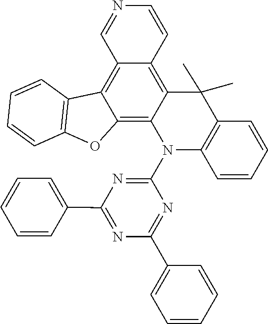 [SMILES: CC1(C)c2ccccc2N(c2nc(-c3ccccc3)nc(-c3ccccc3)n2)c2c1c1ccncc1c1c2oc2ccccc21] | 2.8g 57% |
| | | EX148 | |
| | | MS (m/z, EI⁺): 580.7 | |
| 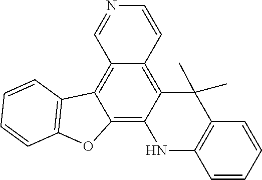 [SMILES: CC1(C)c2ccccc2Nc2c1c1ccncc1c1c2oc2ccccc21] 5f (3.0 g, 8.5 mmol) | 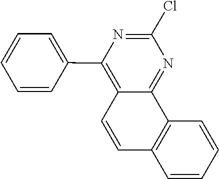 [SMILES: Clc1nc(-c2ccccc2)c2ccc3ccccc3c2n1] (3.0 g, 10.2 mmol) | 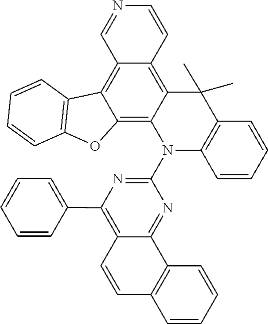 [SMILES: CC1(C)c2ccccc2N(c2nc(-c3ccccc3)c3ccc4ccccc4c3n2)c2c1c1ccncc1c1c2oc2ccccc21] | 3.0 g 59% |
| | | EX154 | |
| | | MS (m/z, EI⁺): 603.7 | |
| 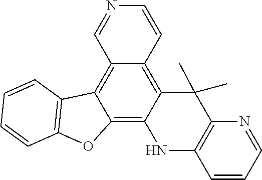 [SMILES: CC1(C)c2ncccc2Nc2c1c1ccncc1c1c2oc2ccccc21] 5g (3.0 g, 8.5 mmol) | 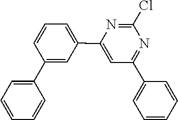 [SMILES: Clc1nc(-c2ccccc2)cc(-c2cccc(-c3ccccc3)c2)n1] (3.5 g, 10.2 mmol) | 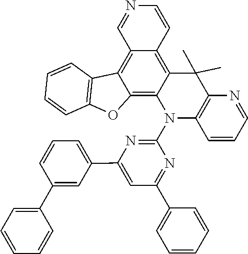 [SMILES: CC1(C)c2ncccc2N(c2nc(-c3ccccc3)cc(-c3cccc(-c4ccccc4)c3)n2)c2c1c1ccncc1c1c2oc2ccccc21] | 3.2 g 58% |
| | | EX167 | |
| | | MS (m/z, EI⁺): 656.8 | |
| 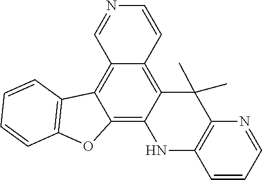 [SMILES: CC1(C)c2ncccc2Nc2c1c1ccncc1c1c2oc2ccccc21] 5g (3.0 g, 8.5 mmol) | 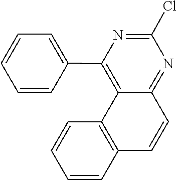 [SMILES: Clc1nc(-c2ccccc2)c2c(ccc3ccccc32)n1] (3.0 g, 10.2 mmol) | 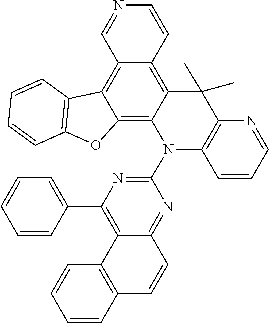 [SMILES: CC1(C)c2ncccc2N(c2nc(-c3ccccc3)c3c(ccc4ccccc43)n2)c2c1c1ccncc1c1c2oc2ccccc21] | 2.9g 57% |
| | | EX171 | |
| | | MS (m/z, EI⁺): 604.7 | |

### EXAMPLE 16

### Synthesis of EX82

### Synthesis of Intermediate 3c

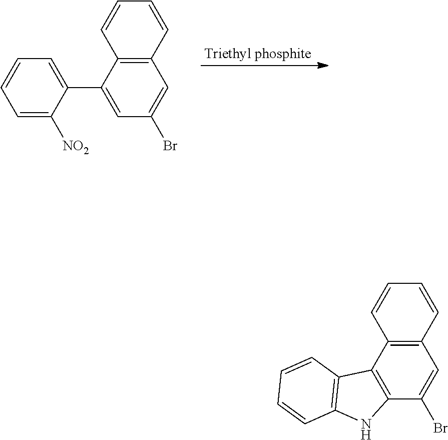 [SMILES: O=[N+]([O-])c1ccccc1-c1cc(Br)cc2ccccc12 ; "Triethyl phosphite" ; Brc1cc2ccccc2c2c1[nH]c1ccccc12]

A mixture of (18.8 g, 57.3 mmole) of Intermediate 1c, and 90 ml of Triethyl phosphite was degassed and placed under nitrogen, and then heated at 160° C. for 16 h. After the reaction finished, the mixture was allowed to cool to room temperature. Then 300 ml of MeOH was added while stirring and the precipitated product was filtered off with suction to give (12.5 g, 74%) of yellow product, which was recrystallized from Toluene.

### Synthesis of Intermediate 3d

Synthesis of Intermediate 3d were according to the synthesis method of Intermediate 3c.

| | | Weight |
|---|---|---|
| Reactant structure | Product structure | Yield |
| 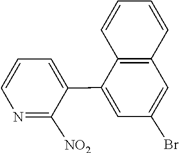 [SMILES: O=[N+]([O-])c1ncccc1-c1cc(Br)cc2ccccc12] 1d (16.4g, 49.8 mmol) | 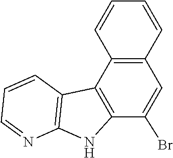 [SMILES: Brc1cc2ccccc2c2c1[nH]c1ncccc12] 3d | 11.1 g 75% |

### Synthesis of Intermediate 4c

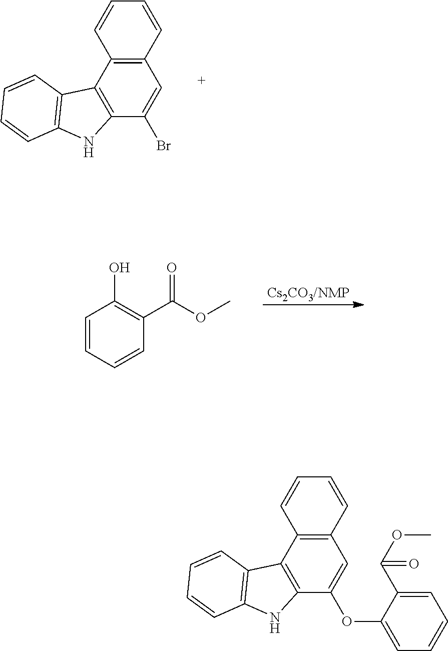

A mixture of (12.5 g, 42.3 mmole) of Intermediate 3c, (7.1 g, 46.6 mmole) of Methyl salicylate, (20.7 g, 63.5 mmole) of Cesium carbonate, 60 ml of N-methyl-2-pyrrolidone was degassed and placed under nitrogen, and then heated at 120° C. overnight. After the reaction finished, the mixture was allowed to cool to room temperature. Then 300 ml of H₂O was added while stirring and the precipitated product was filtered off with suction to give (14.4 g, 93%) of yellow product.

### Synthesis of Intermediate 4d, 4h and 4i

Synthesis of Intermediate 4d, 4h and 4i were according to the synthesis method of Intermediate 4c.

| | | | Weight |
|---|---|---|---|
| Reactant structure | | Product structure | Yield |
| 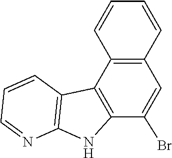 [SMILES: Brc1cc2ccccc2c2c1[nH]c1ncccc12] (10.0 g, 33.6 mmol) | 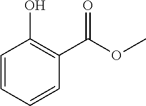 (5.6 g, 37.0 mmol) | 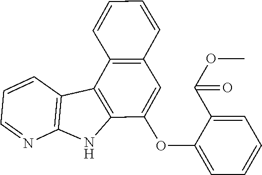 [SMILES: COC(=O)c1ccccc1Oc1cc2ccccc2c2c1[nH]c1ncccc12] 4d | 11.0 g 89% |
| 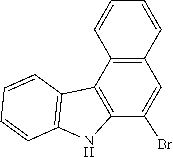 [SMILES: Brc1cc2ccccc2c2c1[nH]c1ccccc12] (10.0 g, 33.7 mmol) | 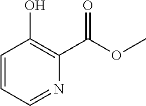 (5.7 g, 37.0 mmol) | 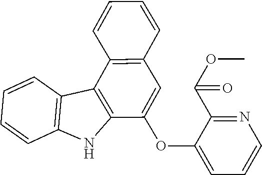 [SMILES: COC(=O)c1ncccc1Oc1cc2ccccc2c2c1[nH]c1ccccc12] 4h | 10.5 g 85% |
| 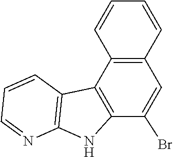 [SMILES: Brc1cc2ccccc2c2c1[nH]c1ncccc12] (10.0 g, 33.6 mmol) | 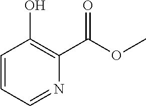 (5.7 g, 37.0 mmol) | 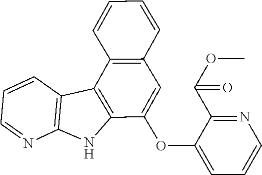 [SMILES: COC(=O)c1ncccc1Oc1cc2ccccc2c2c1[nH]c1ncccc12] 4i | 10.9 g 88% |

### Synthesis of Intermediate 5c, 5d, 5h and 5i

Synthesis of Intermediate5c, 5d, 5h and 5i were according to the synthesis method of Intermediate 5a.

| | | Weight |
|---|---|---|
| Reactant structure | Product structure | Yield |
| 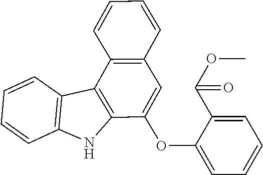 [SMILES: COC(=O)c1ccccc1Oc1cc2ccccc2c2c1[nH]c1ccccc12] | 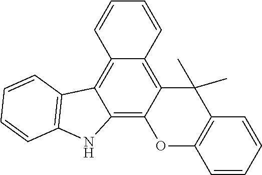 [SMILES: CC1(C)c2ccccc2Oc2c1c1ccccc1c1c2[nH]c2ccccc21] | 8.6 g 63% |
| 4c (14.4 g, 39.2 mmol) | 5c | |
| 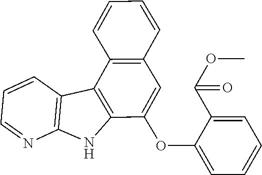 [SMILES: COC(=O)c1ccccc1Oc1cc2ccccc2c2c1[nH]c1ncccc12] | 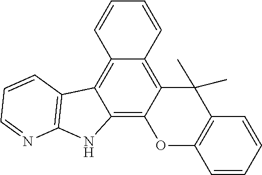 [SMILES: CC1(C)c2ccccc2Oc2c1c1ccccc1c1c2[nH]c2ncccc21] | 6.3 g 60% |
| 4d (11.0 g, 29.9 mmol) | 5d | |
| 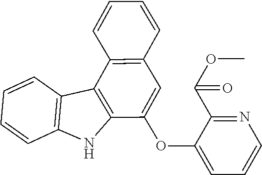 [SMILES: COC(=O)c1ncccc1Oc1cc2ccccc2c2c1[nH]c1ccccc12] | 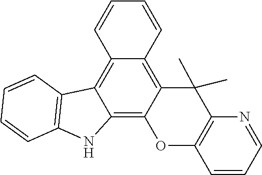 [SMILES: CC1(C)c2ncccc2Oc2c1c1ccccc1c1c2[nH]c2ccccc21] | 6.1 g 61% |
| 4h (10.5 g, 28.5 mmol) | 5h | |
| 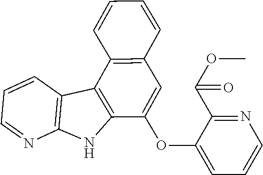 [SMILES: COC(=O)c1ncccc1Oc1cc2ccccc2c2c1[nH]c1ncccc12] | 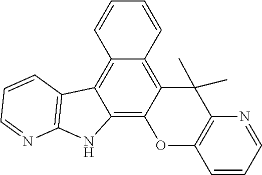 [SMILES: CC1(C)c2ncccc2Oc2c1c1ccccc1c1c2[nH]c2ncccc21] | 5.1 g 49% |
| 4i (10.9 g, 29.5 mmol) | 5i | |

### EXAMPLE 16 to 22

Synthesis of Compound EX82, EX86, EX95, EX104, EX114, EX161, EX165, EX176 and EX180.

Synthesis of Compound EX82, EX86, EX95, EX104, EX114, EX161, EX165, EX176 and EX180 were prepared according to the synthesis method of Compound EX1.

| | | | Weight |
|---|---|---|---|
| Reactant structure | | Product structure | Yield |
| 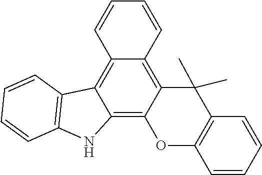 [SMILES: CC1(C)c2ccccc2Oc2c1c1ccccc1c1c2[nH]c2ccccc21] 5c (3.0 g, 8.6 mmol) | 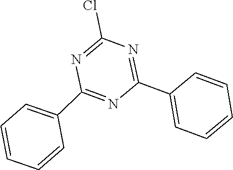 [SMILES: Clc1nc(-c2ccccc2)nc(-c2ccccc2)n1] (2.7 g, 10.3 mmol) | 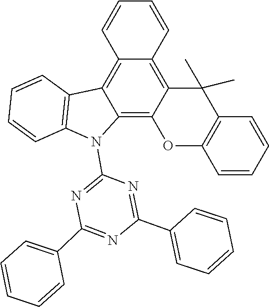 [SMILES: CC1(C)c2ccccc2Oc2c1c1ccccc1c1c3ccccc3n(-c3nc(-c4ccccc4)nc(-c4ccccc4)n3)c21] | 3.4 g 69% |
| | | EX82 | |
| | | MS (m/z, EI⁺): 579.7 | |
| 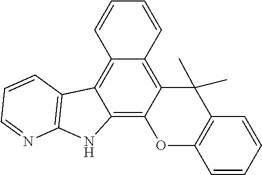 [SMILES: CC1(C)c2ccccc2Oc2c1c1ccccc1c1c2[nH]c2ncccc21] 5d (3.0 g, 8.6 mmol) | 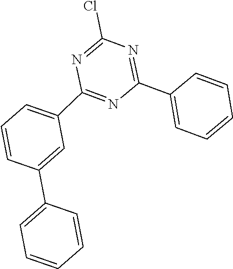 [SMILES: Clc1nc(-c2ccccc2)nc(-c2cccc(-c3ccccc3)c2)n1] (3.5 g, 10.3 mmol) | 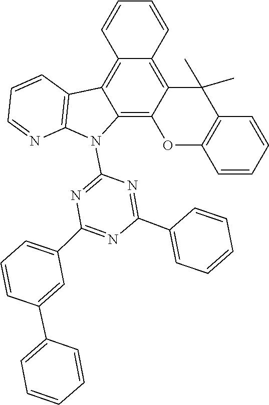 [SMILES: CC1(C)c2ccccc2Oc2c1c1ccccc1c1c3cccnc3n(-c3nc(-c4ccccc4)nc(-c4cccc(-c5ccccc5)c4)n3)c21] | 3.7 g 65% |
| | | EX86 | |
| | | MS (m/z, EI⁺): 656.8 | |
| 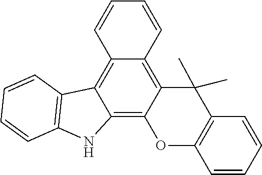 [SMILES: CC1(C)c2ccccc2Oc2c1c1ccccc1c1c2[nH]c2ccccc21] 5c (3.0 g, 8.6 mmol) | 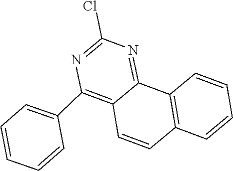 [SMILES: Clc1nc(-c2ccccc2)c2ccc3ccccc3c2n1] (3.0 g, 10.3 mmol) | 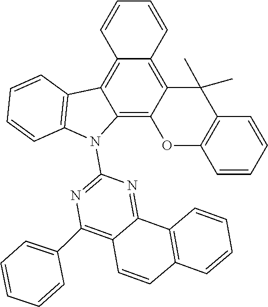 [SMILES: CC1(C)c2ccccc2Oc2c1c1ccccc1c1c3ccccc3n(-c3nc(-c4ccccc4)c4ccc5ccccc5c4n3)c21] | 3.5 g 68% |
| | | EX95 | |
| | | MS (m/z, EI⁺): 602.7 | |
| 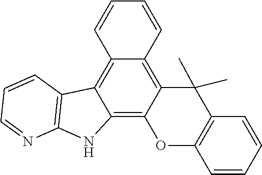 [SMILES: CC1(C)c2ccccc2Oc2c1c1ccccc1c1c2[nH]c2ncccc21] 5d (3.0 g, 8.6 mmol) | 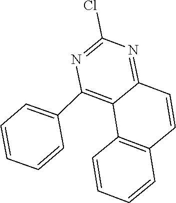 [SMILES: Clc1nc(-c2ccccc2)c2c(ccc3ccccc32)n1] (3.0 g, 10.3 mmol) | 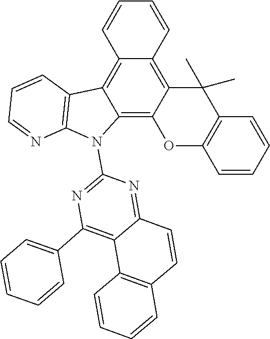 [SMILES: CC1(C)c2ccccc2Oc2c1c1ccccc1c1c3cccnc3n(-c3nc(-c4ccccc4)c4c(ccc5ccccc54)n3)c21] | 3.2 g 61% |
| | | EX104 | |
| | | MS (m/z, EI⁺): 603.8 | |
| 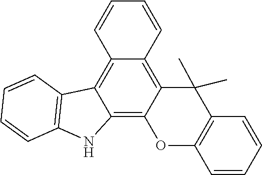 [SMILES: CC1(C)c2ccccc2Oc2c1c1ccccc1c1c2[nH]c2ccccc21] 5c (3.0 g, 8.6 mmol) | 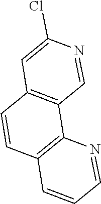 [SMILES: Clc1cc2ccc3cccnc3c2cn1] (2.2 g, 10.3 mmol) | 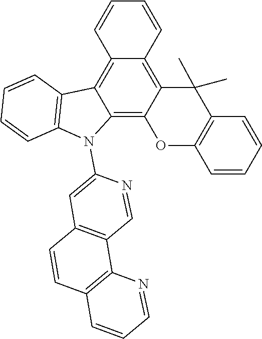 [SMILES: CC1(C)c2ccccc2Oc2c1c1ccccc1c1c3ccccc3n(-c3cc4ccc5cccnc5c4cn3)c21] | 2.9 g 64% |
| | | EX114 | |
| | | MS (m/z, EI⁺): 526.6 | |
| 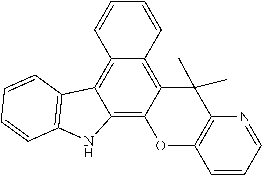 [SMILES: CC1(C)c2ncccc2Oc2c1c1ccccc1c1c2[nH]c2ccccc21] 5h (3.0 g, 8.6 mmol) | 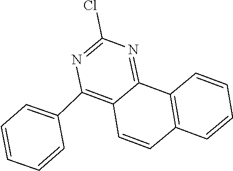 [SMILES: Clc1nc(-c2ccccc2)c2ccc3ccccc3c2n1] (3.0 g, 10.3 mmol) | 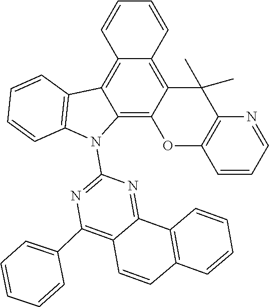 [SMILES: CC1(C)c2ncccc2Oc2c1c1ccccc1c1c3ccccc3n(-c3nc(-c4ccccc4)c4ccc5ccccc5c4n3)c21] | 3.1 g 59% |
| | | EX161 | |
| | | MS (m/z, EI⁺): 603.8 | |
| 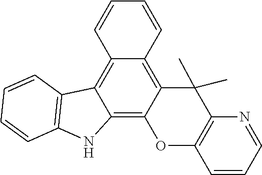 [SMILES: CC1(C)c2ncccc2Oc2c1c1ccccc1c1c2[nH]c2ccccc21] 5h (3.0 g, 8.6 mmol) | 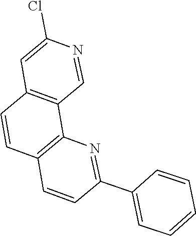 [SMILES: Clc1cc2ccc3ccc(-c4ccccc4)nc3c2cn1] (3.0 g, 10.2 mmol) | 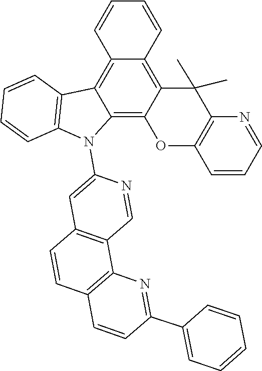 [SMILES: CC1(C)c2ncccc2Oc2c1c1ccccc1c1c3ccccc3n(-c3cc4ccc5ccc(-c6ccccc6)nc5c4cn3)c21] | 3.0 g 57% |
| | | EX165 | |
| | | MS (m/z, EI⁺): 603.8 | |
| 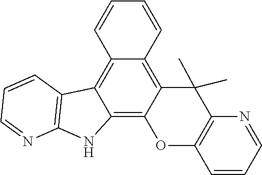 [SMILES: CC1(C)c2ncccc2Oc2c1c1ccccc1c1c2[nH]c2ncccc21] 5i (3.0 g, 8.5 mmol) | 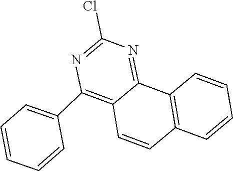 [SMILES: Clc1nc(-c2ccccc2)c2ccc3ccccc3c2n1] (3.0 g, 10.2 mmol) | 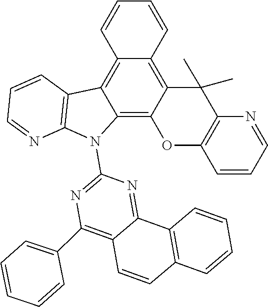 [SMILES: CC1(C)c2ncccc2Oc2c1c1ccccc1c1c3cccnc3n(-c3nc(-c4ccccc4)c4ccc5ccccc5c4n3)c21] | 2.9 g 56% |
| | | EX176 | |
| | | MS (m/z, EI⁺): 604.7 | |

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An organic compound having the following formula (F): 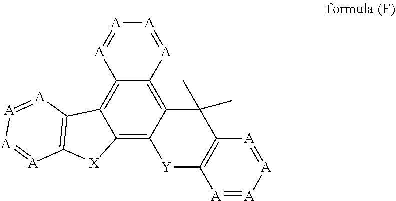
wherein X is O or S if Y is N-L-Z;
wherein Y is O or S if X is N-L-Z;
wherein all A are the same or different at each instance and are independently N or CH;
wherein L represents a single bond or a substituted or unsubstituted divalent arylene group having 6 to 30 ring carbon atoms; and
wherein Z represents a substituted or unsubstituted aryl group having 6 to 60 carbon atoms, or represents a substituted or unsubstituted heteroaryl group having 6 to 60 carbon atoms.

2. The organic compound according to claim 1, wherein Z is selected from the group consisting of the following: 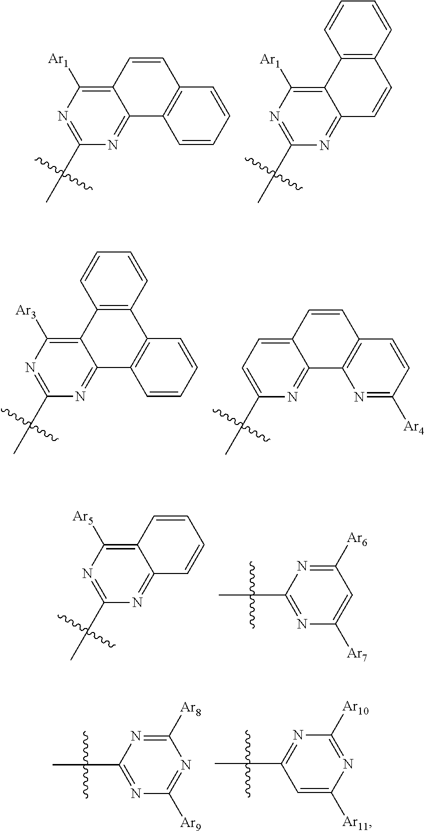
and wherein Ar₁ to Ar₁₁ independently represent a hydrogen atom, a substituted or unsubstituted aryl group having 6 to 30 carbon atoms, or a substituted or unsubstituted heteroaryl group having 6 to 30 carbon atoms.

3. The organic compound according to claim 1, wherein Z is selected from the group consisting of the following:

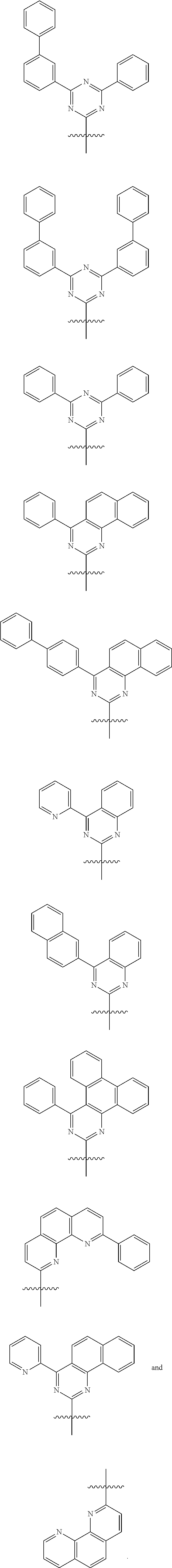

4. The organic compound according to claim 1, wherein the organic compound is selected from the group consisting of the following:

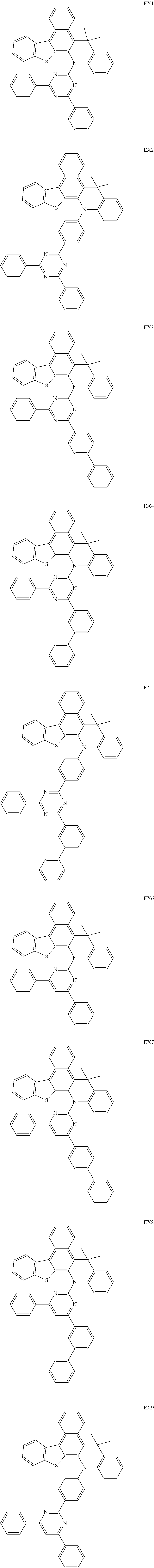

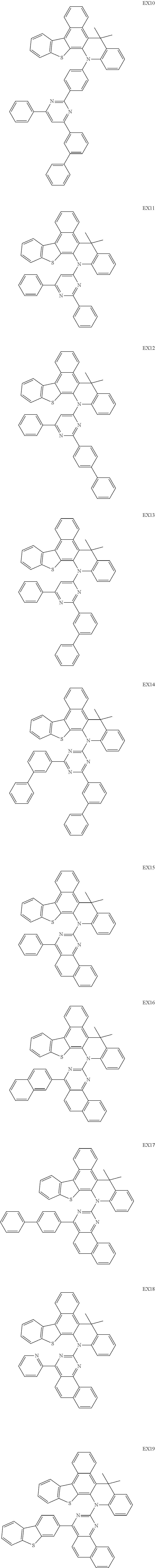

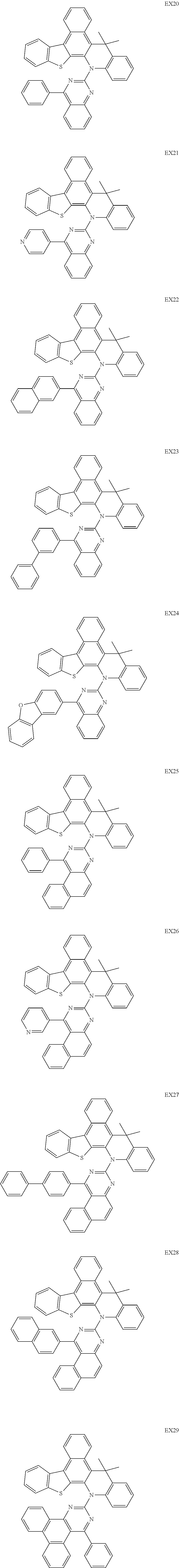

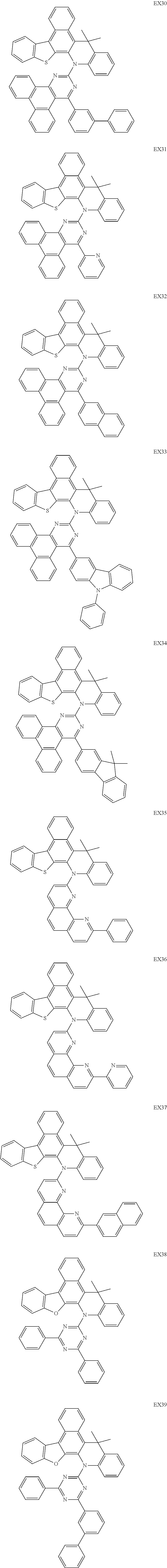

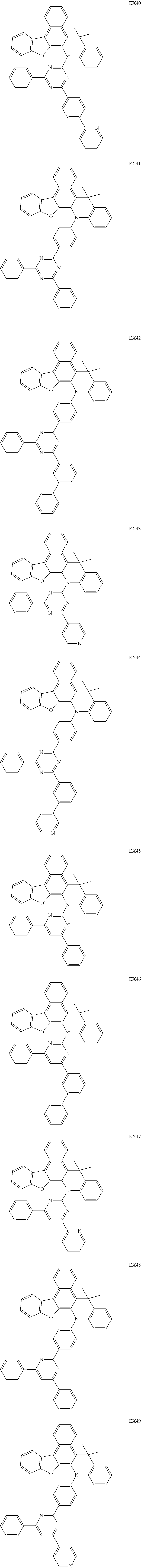

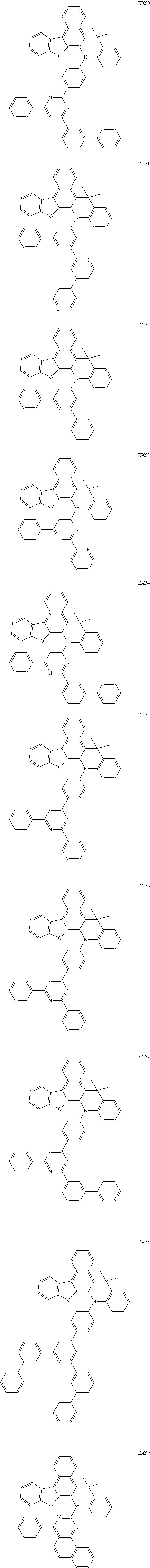

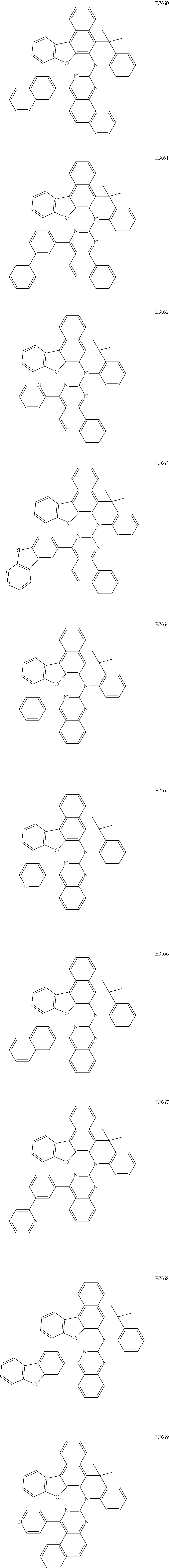

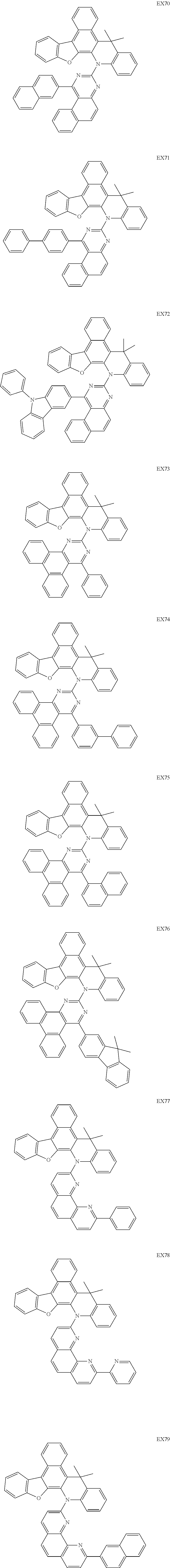

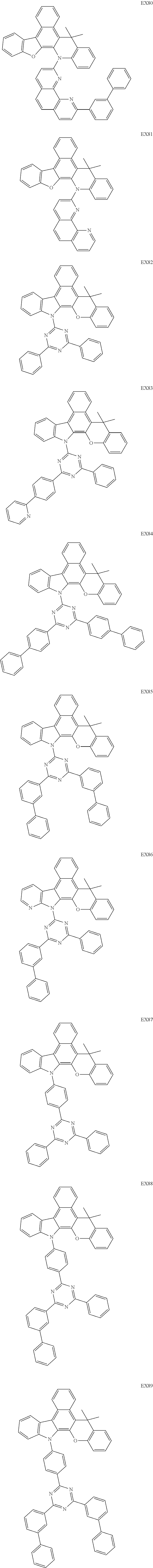

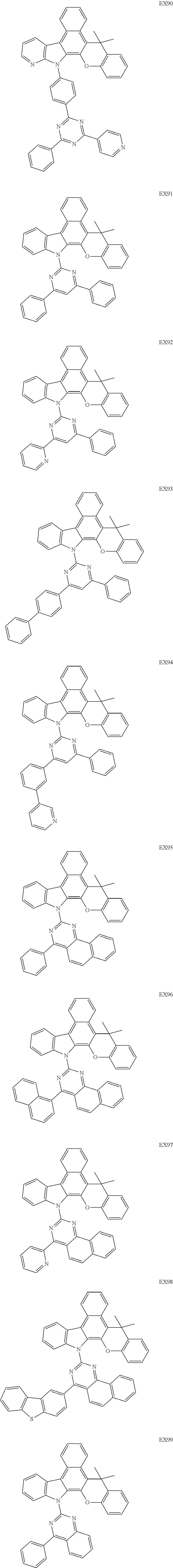

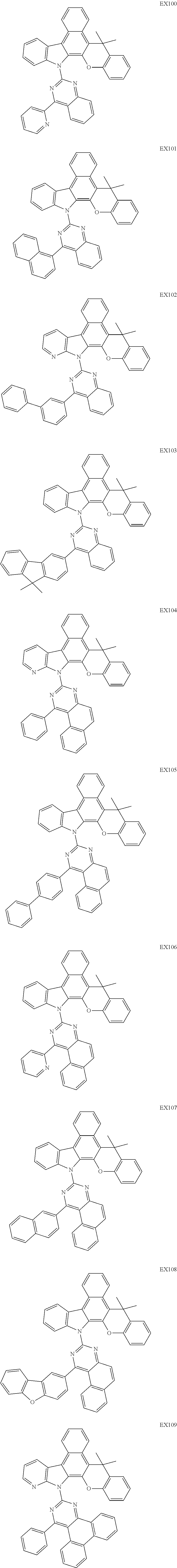

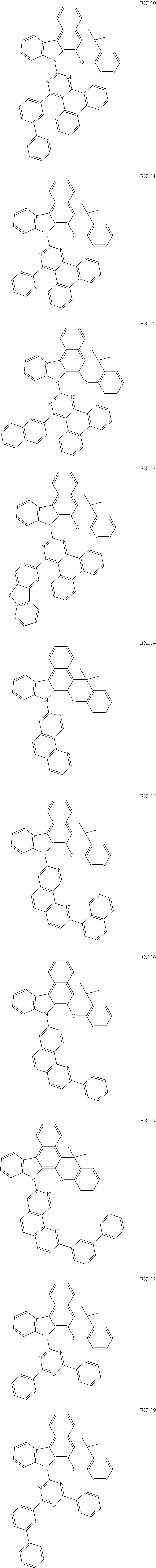

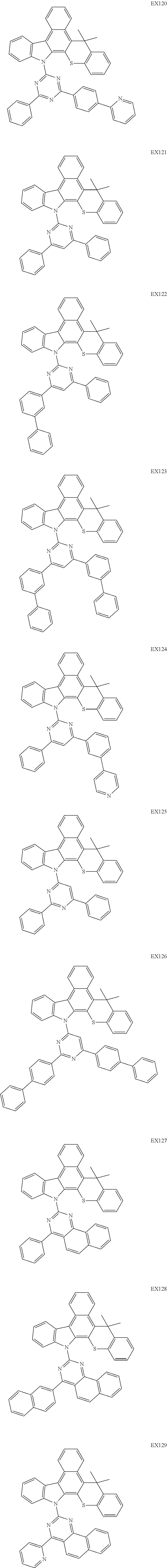

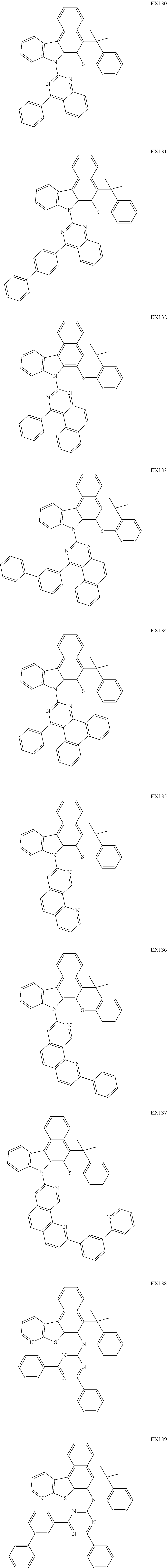

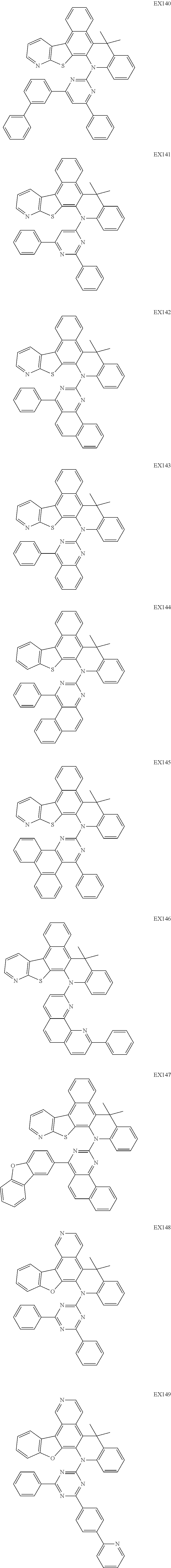

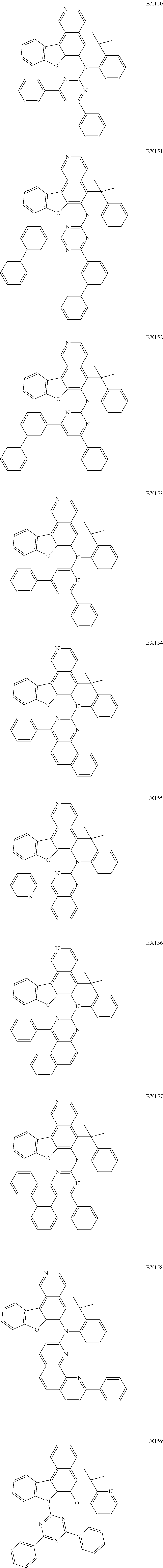

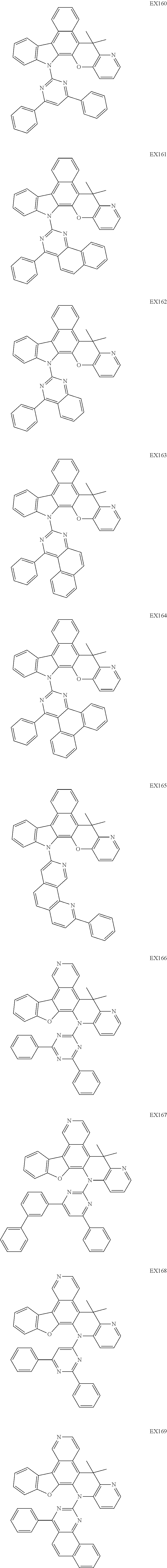

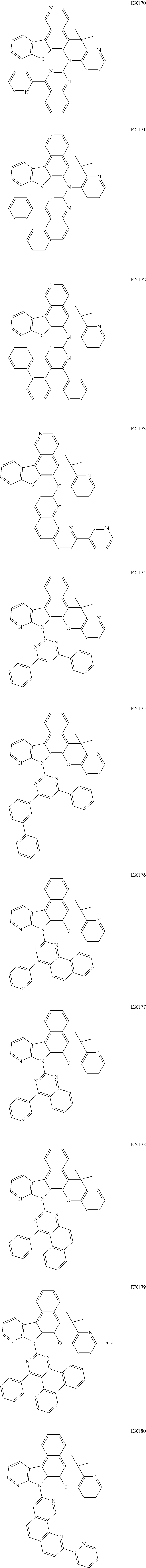

5. An organic electroluminescence device comprising an anode, a cathode and one or more organic layers formed between the anode and the cathode, wherein at least one of the organic layers comprises the organic compound according to claim 1.

6. The organic electroluminescence device according to claim 5, wherein the organic layers comprise an emissive layer having a host, and wherein the organic compound is comprised as the host.

7. The organic electroluminescence device according to claim 6, wherein the host is a fluorescent host.

8. The organic electroluminescence device according to claim 6, wherein the host is a phosphorescent host.

9. The organic electroluminescence device according to claim 5, wherein the organic layers comprise an electron transport layer, and wherein the organic compound is comprised as the electron transport layer.

10. The organic electroluminescence device according to claim 5, wherein the organic layers comprise a hole blocking layer, and wherein the organic compound is comprised as the hole blocking layer.

11. The organic electroluminescent device according to claim 5, wherein the organic electroluminescence device is a lighting panel.

12. The organic electroluminescent device according to claim 5, wherein the organic electroluminescence device is a backlight panel.

13. A organic compound having the following formula (1): 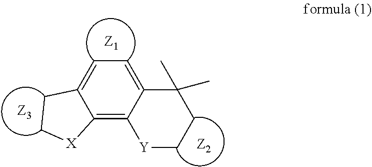
wherein X is O or S if Y is N-L-Z₄; Y is 0 or S if X is N-L-Z₄;
wherein L represents a single bond or arylene;
wherein Z₁ to Z₃ independently represent aromatic hydrocarbons, or aza-aromatic hydrocarbons; and
wherein Z₄ is substituted or unsubstituted heteroaryl having at least two heteroatoms of N.

14. The organic compound according to claim 13, wherein the heteroaryl is selected from the group consisting of benzoquinazolinyl, triazinyl, diazinyl, dibenzoquinazolinyl, pyridinyl, phenylpyridinyl, quinazolinyl, benzodiazinyl, pyridinoquinolinyl, phenyl, biphenyl, and combinations thereof, and

15. The organic compound according to claim 14, wherein the heteroaryl is substituted by phenyl, biphenyl, dibenzofuranyl, dibenzothiophenyl, 9,9-dimethyl-9H-fluorenyl, naphthalenyl, or combinations thereof.

16. The organic compound according to claim 13, wherein Z₄ is polycyclic heteroaryl or monocyclic heteroaryl, wherein the polycyclic heteroaryl has a plurality of aromatic rings.

17. The organic compound according to claim 16, wherein Z₄ is tricyclic heteroaryl.

18. The organic compound according to claim 16, wherein Z₄ is tetracyclic heteroaryl.

19. The organic compound according to claim 16, wherein Z₄ is heteroaryl having only two heteroatoms of N.

20. The organic compound according to claim 13, wherein each of Z₁ to Z₃ represents benzene.
